(19) United States
(12) Patent Application Publication
(10) Pub. No.: US 2022/0387516 A1
(43) Pub. Date: Dec. 8, 2022

(54) FIBROBLAST-DERIVED UNIVERSAL IMMUNOLOGICAL COMPOSITION

(71) Applicant: FIGENE, LLC, Houston, TX (US)

(72) Inventors: Pete O'HEERON, Houston, TX (US); Thomas ICHIM, San Diego, CA (US)

(21) Appl. No.: 17/755,275

(22) PCT Filed: Nov. 2, 2020
(86) PCT No.: PCT/US2020/058492

§ 371 Date: Apr. 26, 2022

Related U.S. Application Data
(60) Provisional application No. 62/929,828, filed on Nov. 2, 2019.

(51) Int. Cl. A61K 35/33; A61K 35/17; A61K 35/545; A61K 35/50; A61K 39/00; A61P 35/00

(52) U.S. Cl. CPC A61K 35/33; A61K 35/17; A61K 35/545; A61K 35/50; A61K 39/00114; A61K 39/001141; A61P 35/00

(57) ABSTRACT

Described are means of generating immunological compositions that are universally applicable for induction of immunity to neoplasia regardless of histological origin of tissue. Certain methods concern fibroblasts that are manipulated or dedifferentiated in a manner to induce expression of tumor associated antigens including cancer testis antigens. These cells are used as a source of antigenic stimuli for creation of a cellular vaccine, and/or an exosome vaccine, and/or a lysate-based vaccine.

### BACKGROUND

This application claims priority to U.S. Provisional Patent Application Serial No. 62/929,828, filed Nov. 2, 2019, which is incorporated by reference herein in its entirety.

### TECHNICAL FIELD

Embodiments of the disclosure include at least the fields of molecular biology, cell biology, immunology, and medicine.

Numerous advances have been made scientifically and medically which resulted in multiple preventive and therapeutic approaches to cancer. Unfortunately, cancer is one of the major causes of death worldwide. In addition to chemotherapy and radiotherapy, manipulation of the immune system in different types of immunotherapies has shown encouraging results in human clinical trials. Different modalities of cancer vaccines have shown some degree of clinical efficacy. Whole tumor cell vaccines, administered in presence of adjuvant and genetically engineered tumor cells that express cytokines. These vaccines have the advantage of expressing relevant tumor-associated antigens shared by the patient's cancer cells. However, one of the disadvantages of these vaccines is the weak antigen presentation, poor ability to stimulate a potent immune response and the potential to cause autoimmune reaction due to non-tumor specific stimulation of immunity against self antigens co-expressed by normal cells.

The use of complex mixtures of whole tumor cells or tumor-derived material does not take advantage of the specificity of treatment that immunotherapy can provide over other forms of therapy. Theoretically, an antibody or T-cell mediated immune response can recognize unique epitopes that are differentially expressed by tumor cells and destroy those cells that express that antigen without affecting normal cells and without the risk of triggering an autoimmune response. To take advantage of specificity, large efforts have been invested in the discovery of tumor associated antigens (TAA). Antigen-specific immunotherapy represents an attractive approach for cancer treatment because of the capacity to eradicate systemic tumors at multiple sites in the body while retaining the exquisite specificity to discriminate between neoplastic and non-neoplastic cells.

One of the major problems in cancer immunotherapy is that the tumor is many times seen as “immune privileged” or belonging to “self”. Cancer employs many of the molecules used by the body in natural conditions of tolerance to accomplish this “masking”. For example, cancer, similar to pregnancy, testicular immune privilege, and ocular immune privilege, uses the same molecules and mechanisms such as TGF-beta, IL-10, FAS-ligand, and activation of suppressor cells such as myeloid suppressor cells and T regulatory cells.

Currently, there is a need to manipulate the tumor, or at least part of the tumor, in order to endow a state of immunogenicity. The triggering of immunogenicity in one tumor has been shown in some cases to systemically break tolerance and result in distant metastasis going into remission, a phenomenon called the abscopal effect. The current disclosure also harnesses the potently innate existing antibody to alphaGal as a means of awaking the immune system out of dormancy to attack tumors and break tolerance.

### BRIEF SUMMARY

The present disclosure concerns compositions and methods for manipulated fibroblasts that are useful in comprising one or more immunological compositions, including an immunological composition for cancer, such as a cancer vaccine. In certain embodiments, the fibroblasts are manipulated to express, produce, and/or present certain antigens or other molecules that may be recognized by an individual's immune system. The fibroblasts may be manipulated by dedifferentiation; endogenous and/or exogenous expression of one or more genes that are useful in expression of one or more antigens; exposure to one or more certain signaling molecules; or other manipulations that may lead to the expression of one or more antigens. The fibroblasts may be selected for based on expression of one or more certain antigens. Certain embodiments comprise the administration of fibroblasts of the disclosure to an individual, such as an individual afflicted by cancer or suspected of having or being at risk for cancer. The fibroblasts administered to the individual may express one or more antigens that are specific for the cancer afflicting the individual.

In some embodiments, fibroblasts are manipulated to express or produce one or more antigens that may not be expressed in fibroblasts that are not manipulated to express or produce one or more antigens. The fibroblasts may be dedifferentiated to express or produce one or more certain antigens. In some embodiments, the fibroblasts may be manipulated to express one or more exogenous antigens, including manipulating fibroblasts to express an exogenous antigen from an expression vector or any other system for exogenous antigen expression, and/or express exogenous genes, including manipulating fibroblasts to express an exogenous gene from an expression vector or any other system for exogenous gene expression. that leads to the expression of one or more certain antigens. In some embodiments, the fibroblasts may be exposed to hypoxic conditions, acidic conditions, cytoplasmic contents from dedifferentiated and/or pluripotent cells, one or more dedifferentiation signals, one or more histone deacetylase inhibitors, one or more DNA methyltransferase inhibitors, one or more GSK-3 inhibitors, a combination thereof, or the like.

In some embodiments, the fibroblasts may be manipulated to express or produce one or more antigens that are present on a tumor including, but not limited to, hTERT, c-myc, k-RAS, CTCFL, AF4/HRX, ABL, ALK, AKT-2, ALK/NPM, AML1, AML1/MTG8, AXL, BCL-2, BCL-3, BCL-6, BCL-XL, BCR/ABL, DBL, DEK/CAN, E2A/PBX1, EGFR, ENL/HRX, ERG/TLS, ERBB, ERBB-2, ETS-1, EWS/FLI-1, FMS, FOS, FPS, GLI, GSP, HOX11, HER2/neu, HST, INT-2, JUN, KIT, KS3, K-SAM, LBC, LCK, LM02/LM01, LYL-1, LYT-10, TSK, TRK, Fos-related antigen 1, LCK, FAP, VEGFR2, NA17, PDGFR-beta, PAP, MAD-CT-2, Tie-2, PSA, protamine 2, legumain, endosialin, prostate stem cell antigen, carbonic anhydrase IX, STn, Page4, proteinase 3, GM3 ganglioside, tyrosinase, MART1, gp100, SART3, RGS5, SSX2, Globoll, Tn, CEA, hCG, PRAME, XAGE-1, AKAP-4, TRP-2, B7H3, sperm fibrous sheath protein, CYP1B1, HMWMAA, sLe(a), MAGE Al, GD2, PSMA, mesothelin, fucosyl GM1, GD3, sperm protein 17, NY-ESO-1, PAX5, AFP, polysialic acid, EpCAM, MAGE-A3, mutant p53, ras, mutant ras, NY-BR1, PAX3, HER2/neu, 0Y-TES1, HPV E6 E7, PLAC1, hTERT, BORIS, ML-IAP, idiotype of b cell lymphoma or multiple myeloma, EphA2, EGFRvIII, cyclin B 1, RhoC, androgen receptor, surviving, MYCN, wildtype p53, LMP2, ETV6-AML, MUC1, BCR-ABL, ALK, WT1, ERG (TMPRSS2 ETS fusion gene), sarcoma translocation breakpoint, STEAP, OFA/iLRP, Chondroitin sulfate proteoglycan 4 (CSPG4), alphaGal, or any other antigen present on a tumor, or a combination thereof.

In some embodiments, the fibroblasts may be administered to an individual having, or at risk of having, cancer of any kind, including solid tumors or hematological malignancies. The fibroblasts may be provided to an individual that is also provided one or more other compositions. In some embodiments, the fibroblasts are administered prophylactically, therapeutically, or both. The administration of fibroblasts may induce an immune response in the individual against the one or more antigens present on the fibroblasts. The immune response may induce immunity to the cancer in the individual. The immune response may induce complement activation, T cell activation, NK cell activation, M1 cell activation, M2 cell suppression, angiogenesis suppression, or a combination thereof in or near the cancer. The immune response may result in the generation of antibodies against the antigens disclosed herein. The antibodies may induce antibody dependent cellular cytotoxicity.

### BRIEF DESCRIPTION OF THE DRAWINGS

FIG. 1 shows dedifferentiated fibroblasts reduce tumor volume in a B16 melanoma xenograft model compared to control, differentiated fibroblasts. Diamonds are control saline; squares are controlled fibroblasts; X is dedifferentiated fibroblast protocol 1, and triangles represent dedifferentiated fibroblast protocol 2.

FIG. 2 shows dedifferentiated fibroblasts reduce tumor volume in a 4T1 breast cancer xenograft model compared to control, differentiated fibroblasts. Diamonds are control saline; squares are controlled fibroblasts; X is dedifferentiated fibroblast protocol 1, and triangles represent dedifferentiated fibroblast protocol 2.

FIG. 3 demonstrates fold difference of MAGE expression over housekeeping GAPDH in the presence of 5-azacytidine, lithium, or a combination thereof. In the groupings of four bars, from left to right is as follows: control, 5-azacytidine, lithium, and a combination of 5-azacytidine and lithium.

### DETAILED DESCRIPTION

### I. Definitions

Unless defined differently, all technical and scientific terms used herein have the same meanings as commonly understood by one of skill in the art to which the disclosed disclosure belongs. In particular, the following terms and phrases have the following meaning.

Unless defined otherwise, all technical and scientific terms used herein have the same meaning as commonly understood by one of ordinary skill in the art to which the disclosure pertains. The articles “a” and “an” are used herein to refer to one or to more than one (i.e., to at least one) of the grammatical object of the article. By way of example, “an element” means one element or more than one element.

“About” as used herein, when referring to a measurable value such as an amount, a temporal duration, and the like, is meant to encompass variations of +/−20% or in some instances +/−10%, or in some instances +/−5%, or in some instances +/−1%, or in some instances +/−0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, “adjuvant” refers to a substance that is capable of enhancing, accelerating, or prolonging an immune response when given with a vaccine immunogen.

“Administering” as used herein, refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

As used herein, “agonist” refers to a substance that promotes (induces, causes, enhances or increases) the activity of another molecule or a receptor. The term agonist encompasses substances that bind a receptor (e.g., an antibody, a homolog of a natural ligand from another species) and substances that promote receptor function without binding thereto (e.g., by activating an associated protein).

The term “anergy” and “unresponsiveness” refer generally to the unresponsiveness to an immune cell to stimulation, for example, stimulation by an activation receptor or cytokine. The anergy may occur due to, for example, exposure to an immune suppressor or exposure to an antigen in a high dose. Such anergy is generally antigen-specific, and continues even after completion of exposure to a tolerized antigen. For example, the anergy in a T cell and/or NK cell is characterized by failure of production of cytokine, for example, interleukin (IL)-2. The T cell anergy and/or NK cell anergy occurs in part when a first signal (signal via TCR or CD-3) is received in the absence of a second signal (costimulatory signal) upon exposure of a T cell and/or NK cell to an antigen.

As used herein, “antagonist” or “inhibitor” refers to a substance that partially or fully blocks, inhibits, or neutralizes a biological activity of another molecule or receptor.

The term “antibody” is meant to include both intact molecules as well as functional fragments thereof that include the antigen-binding site. Whole antibody structure is often given as H₂L₂ and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure. The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The antibodies disclosed according to the disclosure may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect that can be manifested by a decrease in tumor volume, a decrease in the number of tumor cells, a decrease in the number of metastases, an increase in life expectancy, and/or amelioration of various physiological symptoms associated with the cancerous condition. An “anti-tumor effect” can also be manifested by the ability of the peptides, polynucleotides, cells and/or antibodies of the disclosure in prevention of the occurrence of tumor in the first place or delay in its onset or reduction in its severity or in inhibiting its metastasis.

The term “auto-antigen” means, in accordance with the present disclosure, any self-antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

As used herein, the term “autologous” is meant to refer to any material derived from the same individual to which it is later to be re-introduced into the individual.

As used herein, the term “allogeneic” refers to a graft derived from a different animal of the same species.

As used herein, “cancer” is defined as disease characterized by the rapid and uncontrolled growth of aberrant cells. Cancer cells can spread locally or through the bloodstream and lymphatic system to other parts of the body. “Cancer” refers to all types of cancer or neoplasm or malignant tumors found in animals, including for example leukemias, lymphomas, carcinomas, melanomas, and sarcomas. Non-limiting examples of cancers are cancer of the brain, skin, bladder, breast, cervix, colon, head and neck, kidney and renal pelvis, lung, liver, non-small cell lung, mesothelioma, ovary, pancreas, prostate, sarcoma, stomach, thyroid, endometrium, uterus and medulloblastoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

The term “carcinoma” refers to a malignant new growth made up of epithelial cells that may infiltrate the surrounding tissues, resist physiological and non-physiological cell death signals, and/or give rise to metastases. Exemplary carcinomas include, for example, cinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti.

The term “CD137” refers to a TNFR-family member with costimulatory function. CD137 is also called 4-1BB or TNFSFR9. It was originally identified as an inducible molecule expressed on activated mouse and human CD8+ and CD4+ T-cells. CD137 signaling regulates T-cell proliferation and survival, particularly within the T-cell memory pool and can upregulate Bcl-X_{L} anti-apoptotic protein expression and supports CD8+ T-cell expansion.

As used herein, “co-administration” refers to administration of two or more compositions to the same subject or individual during a treatment period. The two or more compositions may be encompassed in a single formulation and thus be administered simultaneously. Alternatively, the two or more compositions may be in separate physical formulations and administered separately, either sequentially or simultaneously to the subject or individual. The term “administered simultaneously” or “simultaneous administration” means that the administration of the first agent and that of a second agent overlap in time with each other, while the term “administered sequentially” or “sequential administration” means that the administration of the first agent and that of a second agent does not overlap in time with each other.

“Costimulatory ligand” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, CD7, B7-1 (CD80), B7-2 (CD86), programmed death (PD) L1, PD-L2, 4-1BB ligand, OX40 ligand, inducible costimulatory ligand (ICOS-L), intercellular adhesion molecule (ICAM), CD30 ligand, CD40, CD70, CD83, human leukocyte antigen G (HLA-G), MHC class I chain-related protein A (MICA), MHC class I chain-related protein B (MICB), herpes virus entry mediator (HVEM), lymphotoxin beta receptor, 3/TR6, immunoglobulin-like transcript (ILT) 3, ILT4, an agonist or antibody that binds Toll ligand receptor and a ligand that specifically binds with B7-H3. A co-stimulatory ligand includes, without limitation, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, tumor necrosis factor superfamily member 14 (TNFSF14 or LIGHT), natural killer cell receptor C (NKG2C), B7-H3, and a ligand that specifically binds with CD83.

“Cytokine,” as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p′70, IL-15, and interferon (IFN) gamma. Examples of pro-inflammatory cytokines include, but are not limited to, IL-la, IL-lb, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

The term “enhanced function of a T cell”, “enhanced cytotoxicity” and “augmented activity” (for example, such as augmentation of immunogenicity) means that the effector function of the T cell and/or NK cell is improved. The enhanced function of the T cell and/or NK cell, which does not limit the present disclosure, includes an improvement in the proliferation rate of the T cell and/or NK cell, an increase in the production amount of cytokine, and/or an improvement in cytotoxicity. Further, the enhanced function of the T cell and/or NK cell includes cancellation and suppression of tolerance of the T cell and/or NK cell in the suppressed state such as the anergy (unresponsive) state, or the rest state, that is, transfer of the T cell and/or NK cell from the suppressed state into the state where the T cell and/or NK cell responds to stimulation from the outside.

The term “expression” means generation of mRNA by transcription from nucleic acids such as genes, polynucleotides, and oligonucleotides, generation of a protein or a polypeptide by transcription from mRNA. “Expression” may also refer to an increase in abundance of an mRNA and/or protein or polypeptide. Expression may be detected by means including RT-PCR, Northern Blot, in situ hybridization, Western blotting, flow cytometry, ELISA, immunohistochemistry, or other detection methods known in the art. “Suppression of expression” refers to a decrease of a transcription product or a translation product in a significant amount as compared with the case of no suppression. The suppression of expression herein may be, for example, a decrease of a transcription product or a translation product in an amount of 30% or more, 50% or more, 70% or more, 90% or more.

As used herein, “ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current disclosure. Although the word “lymphocyte” is used, this also includes heterogenous cells that have been expanded during the ex vivo culturing process including dendritic cells, NKT cells, gamma delta T cells, and various other innate and adaptive immune cells.

The term “immune response” refers to any detectable response to a particular substance (such as an antigen or immunogen) by the immune system of a host vertebrate animal, including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include, but are not limited to, an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+T cell) activation, NK cell activation, B cell activation (e.g., antibody generation and/or secretion), binding of an immunogen (e.g., antigen (e.g., immunogenic polypolypeptide)) to an MHC molecule, induction of a cytotoxic T lymphocyte (CTL) response, induction of a B cell response (e.g., antibody production), expansion (e.g., growth of a population of cells) of cells of the immune system (e.g., T cells and B cells), increased processing and presentation of antigen by antigen presenting cells, or a combination thereof. The term “immune response” also encompasses any detectable response to a particular substance (such as an antigen or immunogen) by one or more components of the immune system of a vertebrate animal in vitro, in vivo, ex vivo, or a combination thereof.

As used herein, the term “immunity” refers the capability of immune cells to activate or trigger an immune response against one or more specific molecules, including one or more specific antigens.

As used herein, the term “immunogenic composition” refers to any composition that can produce an immune response of any kind in an individual.

The term “immunoglobulin” or “Ig”, as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “leukemia” refers broadly to progressive, malignant diseases of the hematopoietic organs/systems and may be generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

The term “lymphocyte” comprises T cells, B cells, NK cells, NK T cells, and Lymphokine Activated Killer (LAK) cells. T-lymphocytes possess T-cell receptors, B-lymphocytes, possess B cell receptors and produce antibodies, Tumor Infiltrating Lymphocytes (TIL) are isolated from tumors and possess some degree of reactivity towards the tumor, cytotoxic T lymphocytes (CTL) are lymphocytes of the CD8 lineage usually and possess ability to kill cells through perforin and/or granzymes. CTL isolation means are described in numerous references including U.S. Pat. Nos. 6,805,861 and 6,531,451. Any one lymphocyte produces one type of TCR or antibody. Each TCR or antibody has specificity for one particular epitope, or antigen binding site, on its cognate antigen. Specific TCRs or antibodies are encoded by genes that are formed from the rearrangement of DNA in a lymphocyte stem cell that encodes the constant (“C”), joining (“J”), variable (“V”) regions, and possibly diversity (“D”) regions of the TCR or antibody. Mammals typically possess one-hundred thousand to one-hundred million lymphocytes of different specificities. Upon stimulation of lymphocytes by an antigen, those lymphocytes specific for the antigen undergo clonal amplification. T lymphocytes are formed in the bone marrow, migrate to and mature in the thymus and then enter the peripheral blood and lymphatic circulation. T lymphocytes are subdivided into three distinct types of cells: helper T cells, suppressor T cells, and cytotoxic T cells. T lymphocytes, unlike B lymphocytes, do not produce antibody molecules, but express a heterodimeric cell surface receptor that recognizes peptide fragments of antigenic proteins that are attached to proteins of the major histocompatibility complex (MHC) and expressed on the surfaces of target cells. T lymphocytes include tumor-infiltrating lymphocytes. Cytotoxic T lymphocytes (CTL) are well known in the art and are typically of the CD3+CD8+CD4− phenotype. They typically lyse cells that display fragments of foreign antigens associated with class I MHC molecules on their cell surfaces. CTL typically recognize normal cells expressing antigens after infection by viruses or other pathogens; and tumor cells that have undergone transformation and are expressing mutated proteins or are over-expressing normal proteins. Natural Killer (NK) cells are well known in the art. NK cells are a subset of lymphocytes active in the immune system and representing an average 15% of mononuclear cells in human peripheral blood. Among the surface markers used to identify human NK cells is a receptor binding with low affinity to the Fc fragment of IgG antibodies, such as Fc-.gamma. receptor III or CD16 antigen. NK cells have been demonstrated to play an important role in vivo in the defense against tumors, tumor metastases, virus infection, and to regulate normal and malignant hematopoiesis. Lymphokine-activated killer (LAK) cells are well known in the art and are a cytotoxic population of cells which are capable of lysing autologous tumor cells and NK-cell resistant tumor cell lines. Precursors of LAK cells belong to the subpopulation of “null” lymphocytes that bear neither T nor B cell surface markers. In the human these precursor cells are widely found in peripheral blood, lymph nodes, bone marrow and the thoracic duct. Purification of LAK cells, and their generation are described in U.S. Pat. Nos. 5,002,879, 4,849,329 and 4,690,915.

The term “melanoma” refers to tumors that may arise from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

The term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as a mammal including a human.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

As used herein, “parenteral” administration of an immunogenic composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any animal, such as a mammal, or cells thereof whether in vitro or in situ, amenable to the methods described herein. In certain non-limiting embodiments, the patient, subject or individual is a human.

The term “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

In accordance with the disclosure herein, the term “reprogramming” means remodeling, in particular erasing and/or remodeling, epigenetic marks of a cell such as DNA methylation, histone methylation or activating genes by inducing transcription factor signal systems, such as for Oct4. In particular, the reprogramming of the present disclosure provides at least one dedifferentiated and/or rejuvenated cell, in particular provides a cell having the characteristic of a multipotent, in particular pluripotent stem cell. Thus, in case the cell to be reprogrammed is cells which already have a multipotent or pluripotent character, the present disclosure may maintain these cells by the reprogramming of the present disclosure in their multi- or pluripotent state for a prolonged period of time. In case the cells to be reprogrammed are in an aged or differentiated state, the present disclosure allows the dedifferentiation into a multipotent or pluripotent stem cell. In a particular embodiment, multipotent cells may be reprogrammed to become pluripotent cells. In some embodiments of the disclosure, the cells are particularly fibroblasts that are to be reprogrammed.

The term “sarcoma” generally refers to a tumor that is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “synthetic antibody” as used herein, is meant an antibody that is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “T cell” is also referred to as T lymphocyte, and generally refers to a thymus-derived cell among lymphocytes involved in an immune response. The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an αβT cell expressing TCR α and β chains, and a γδT cell expressing TCR γ and δ chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used in the present disclosure. These cells may have been activated by cytokine such as IL-2 in vivo or ex vivo. Any cells collected from a living body, or cells obtained via ex vivo culture, for example, a T cell population obtained by the method of the present disclosure as it is, or obtained by freeze preservation, can be used.

The term “therapeutically effective amount” or “effective amount” means a dosage sufficient to treat, inhibit, or alleviate one or more symptoms of a disease state being treated or to otherwise provide a desired pharmacologic and/or physiologic effect, especially enhancing T cell response to a selected antigen. The precise dosage will vary according to a variety of factors such as subject-dependent variables (e.g., age, immune system health, etc.), the disease, and the treatment being administered. The terms “individual”, “host”, “subject”, and “patient” are used interchangeably herein, and refer to a mammal, including, but not limited to, primates, for example, human beings, as well as rodents, such as mice and rats, and other laboratory animals.

As used herein, “treating a cancer”, “inhibiting cancer”, and/or “reducing cancer growth” refer to inhibiting or preventing oncogenic activity of cancer cells. Oncogenic activity can comprise migration, invasion, drug resistance, cell survival, anchorage-independent growth, non-responsiveness to cell death signals, angiogenesis, excessive growth, excessive replication, or combinations thereof of the cancer cells. The terms “cancer”, “cancer cell”, “tumor”, and “tumor cell” are used interchangeably herein and refer generally to a group of diseases characterized by uncontrolled, abnormal growth of cells (e.g., a neoplasia). In some forms of cancer, the cancer cells can spread locally or through the bloodstream and lymphatic system to other parts of the body (“metastatic cancer”).

The term “treatment regimen” refers to a treatment of a disease or a method for achieving a desired physiological change, such as increased or decreased response of the immune system to an antigen or immunogen, such as an increase or decrease in the number or activity of one or more cells, or cell types, that are involved in such response, wherein said treatment or method comprises administering to an animal, such as a mammal, especially a human being, a sufficient amount of one or two or more chemical agents or components of said regimen to effectively treat a disease or to produce said physiological change, wherein said chemical agents or components are administered together, such as part of the same composition, or administered separately and independently at the same time or at different times (i.e., administration of each agent or component is separated by a finite period of time from one or more of the agents or components) and where administration of said one or more agents or components achieves a result greater than that of any of said agents or components when administered alone or in isolation.

As used herein, the term “vaccine” is used broadly to define a composition comprising cells, molecules, antigens, or the like that can induce an immune reaction or immunity in an individual. A vaccine may comprise cells of the present disclosure, including fibroblasts. A vaccine may be combined with other molecules, cells, antigens, adjuvants, immune stimulatory compositions, or the like to modulate the efficacy of the vaccine.

### II. Cells of the Disclosure

Some embodiments of the disclosure provide manipulated fibroblasts that express or produce one or more antigens. In certain embodiments, the cellular membrane of manipulated fibroblasts comprises one or more antigens, although in other cases the antigen is exogenous, such as secreted. The fibroblasts may be from any source. In particular aspects, the fibroblast cells are isolated from nature or are obtained commercially or are obtained from a donor or an individual in need of treatment, or a mixture thereof, for example. The fibroblasts may be autologous with respect to the recipient (obtained from the same host) or allogeneic with respect to the recipient. In addition, the fibroblasts may be xenogeneic to the recipient (obtained from an animal of a different species). The fibroblasts may be syngeneic (genetically similar or identical and hence immunologically compatible) with respect to the recipient.

There are several methods known in the art for the generation of fibroblasts or obtaining them. In some embodiments, the fibroblasts are from omentum, bone marrow, placenta, peripheral blood, cord blood, Wharton's jelly, cerebral spinal fluid, cancer-associated, foreskin, skin, a combination thereof, or any other tissue sufficiently abundant in fibroblasts. In some embodiments, fibroblasts are generated according to protocols previously utilized for treatment of patients utilizing bone marrow-derived MSCs. Specifically, bone marrow is aspirated (10-30 mL) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2×10⁷ cells/ml. Subsequently the cells are centrifuged at approximately 900 g for approximately 30 min or a time period sufficient to achieve separation of mononuclear cells from debris and erythrocytes. The cells are then washed with PBS and plated at a density of approximately 1×10⁶ cells per ml in 175 cm² tissue culture flasks in DMEM with 10% FCS with flasks subsequently being loaded with a minimum of 30 million bone marrow mononuclear cells. The fibroblasts are allowed to adhere for 72 h followed by media changes every 3-4 days. Adherent cells are removed with 0.05% trypsin-EDTA and re-plated at a density of approximately 1×10⁶ per 175 cm².

In some embodiments, the fibroblast cells may be cultured for at least between about 10 days and about 40 days, for at least between about 15 days and about 35 days, for at least between about 15 days and 21 days, such as for at least about 15, 16, 17, 18, 19 or 21 days. In some embodiments, the stem cells of the disclosure may be cultured for no longer than 60 days, or no longer than 50 days, or no longer than 45 days. The fibroblast cells may be cultured 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 or more days. The tissue explants and fibroblasts may be cultured in the presence of a liquid culture medium. Typically, the medium may comprise a basal medium formulation as known in the art. Many basal media formulations can be used to culture fibroblasts herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the fibroblasts cultured. In some embodiments, a culture medium formulation may be explants medium (CEM) which is composed of IMDM supplemented with 10% fetal bovine serum (FBS, Lonza), 100 U/ml penicillin G, 100 μg/ml streptomycin and 2 mmol/L L-glutamine (Sigma-Aldrich). Other embodiments may employ further basal media formulations, such as chosen from the ones above.

For use in the fibroblast culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated (e.g., FBS). As described, the present inventors have realized that by culturing tissue explants and fibroblast cells for time durations as defined above, such as using media compositions as described above, a progenitor or stem cell or fibroblast of the disclosure emerges and proliferates. In some embodiments, fibroblast cells of the present disclosure are identified and characterized by their expression of specific marker proteins, such as cell-surface markers. Detection and isolation of these cells can be achieved, e.g., through flow cytometry, ELISA, and/or magnetic beads. Reverse-transcription polymerase chain reaction (RT-PCR) can also be used to monitor changes in gene expression in response to differentiation. Methods for characterizing fibroblasts of the present disclosure are provided herein. In certain embodiments, the marker proteins used to identify and characterize the stem cells or fibroblasts are selected from the list consisting of c-Kit, Nanog, Sox2, Hey1Vimentin, Cyclin D2, Snail, E-cadherin, Nkx2.5, GATA4, CD105, CD90, CD29, CD73, Wt1, CD34, CD45, and a combination thereof.

In another embodiment of the method of producing fibroblasts of the disclosure, a closed system is used for generating and expanding the fibroblast of the disclosure from bone marrow of normal or other donors. This closed system is a device to functionally expand cells ex vivo. In one specific embodiment, the closed system comprises: 1. a central expansion unit, such as constructed similarly to bioreactors with compressed (within a small unit), but extended growth surfaces; 2. media bags that can be sterilely connected to the expansion unit (e.g. by welding tubes between the unit and the bags) for cell feeding; and 3. Electronic devices to operate automatically the medium exchange, gas supply and temperature. The advantages of the closed system in comparison to conventional flask tissue culture are the construction of a functionally closed system, i.e. the cell input and media bags, are sterile welded to the system. This minimizes the risk of contamination with external pathogens and therefore may be highly suitable for clinical applications. Furthermore, this system can be constructed in a compressed form with consistently smaller cell culture volumes but preserved growth area. The smaller volumes allow the cells to interact more directly with each other which creates a culture environment that is more comparable to the in vivo situation of the bone marrow niche. Also the closed system saves costs for the media and the whole expansion process. The construction of the closed system may involve two sides: the cells are grown inside of multiple fibers with a small medium volume. In some embodiments, the culture media contains growth factors for growth stimulation, and medium without expensive supplements is passed outside the fibers. The fibers are designed to contain nanopores for a constant removal of potentially growth-inhibiting metabolites while important growth-promoting factors are retained in the growth compartment.

Fibroblasts can be isolated and expanded in culture in vitro to obtain sufficient numbers of cells for use in the methods and/or the manufacture of compositions described herein. For example, fibroblasts may be isolated, for example from human bone marrow, and cultured in complete medium (DMEM low glucose containing 4 mM L-glutamine, 10% FBS, and 1% penicillin/streptomycin) in hanging drops, on non-adherent dishes, or any similar culture apparatus. The disclosure, however, should in no way be construed to be limited to any one method of isolating and/or to any culturing medium. Rather, any method of isolating and any method of culturing should be construed to be included in the present disclosure.

Any medium capable of supporting fibroblasts in vitro may be used to culture the fibroblasts. Media formulations that can support the growth of fibroblasts include, but are not limited to, Dulbecco's Modified Eagle's Medium (DMEM), alpha modified Minimal Essential Medium (α, and Roswell Park Memorial Institute Media 1640 (RPMI Media 1640) and the like. The media and conditions for culture of fibroblasts are known in the art. Typically, up to 20% fetal bovine serum (FBS) or 1-20% horse serum is added to the above medium in order to support the growth of fibroblasts. A defined medium, however, also can be used if the growth factors, cytokines, and hormones necessary for culturing fibroblasts are provided at appropriate concentrations in the medium. Media useful in the methods of the disclosure may comprise one or more compounds of interest, including, but not limited to, antibiotics, mitogenic compounds, or differentiation compounds useful for the culturing of fibroblasts. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing fibroblasts. Rather, any method of isolating and culturing fibroblasts should be construed to be included in the present disclosure.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular embodiments, fibroblasts are cultured in the cell culture system, which is a cell culture system, comprising a cell culture medium, preferably in a culture vessel, in particular a cell culture medium supplemented with a substance suitable and determined for protecting the cells from in vitro aging and/or inducing in an unspecific or specific reprogramming.

Furthermore, in the fibroblast culture procedure of some embodiments of the present disclosure, the cell culture medium comprises, optionally in combination with one or more of the substances specified herein, at least one transient proteolysis inhibitor. The use of at least one proteolysis inhibitor in the cell culture medium of the present disclosure increases the time the reprogramming proteins derived from the mRNA or any endogenous genes will be present in the cells and thus facilitates in an even more improved way the reprogramming by the transfected mRNA derived factors described herein. The present disclosure encompasses an embodiment that uses, as a transient proteolysis inhibitor, a protease inhibitor, a proteasome inhibitor and/or a lysosome inhibitor. In some embodiments, the proteosome inhibitor is selected from the group consisting of MG132, TMC-95A, TS-341 and MG262, and a combination thereof. In some embodiments, the protease inhibitor is selected from the group consisting of aprotinin, G-64, leupeptine-hemisulfat, and a combination thereof. In some embodiments, the lysosomal inhibitor is ammonium chloride.

In some embodiments, a cell culture medium comprising at least one transient inhibitor of mRNA degradation is used. The use of a transient inhibitor of mRNA degradation increases the half-life of the reprogramming factors. In another embodiment of the present disclosure, a condition suitable to allow translation of the transfected reprogramming mRNA molecules in the cells is an oxygen content in the cell culture medium from 0.5 to 21%. More particular, and without being bound to the theory, oxygen is used to further induce or increase Oct4 by triggering Oct4 via HIF-1α, in these situations concentrations of oxygen lower than atmospheric concentration may be used, and can be ranging from 0.1% to 10%. In some embodiments, conditions that are suitable to support reprogramming of the cells by the mRNA molecules in the cells are selected; more particularly, these conditions require a temperature from 30 to 38° C., including from 31 to 37° C., including from 32 to 36° C.

The glucose content of the medium, in some embodiments, is below 4.6 g/l, including below 4.5 g/l, including below 4 g/l, including below 3 g/l, including below 2 g/l, including being 1 g/l. DMEM media containing 1 g/l glucose may be used for the present disclosure and are commercially available as “DMEM low glucose” from companies such as PAA, Omega Scientific, Perbio and Biosera. More particular, and without being bound to the theory, high glucose conditions may adversely support aging of cells (methylation, epigenetics) in vitro which may render the reprogramming difficult. In a furthermore preferred embodiment of the present disclosure the cell culture medium contains glucose in a concentration from 0.1 g/l to 4.6 g/l, including 0.5 g/l to 4.5 g/l, and including 1 g/l to 4 g/l.

The fibroblasts of the disclosure may be cultured in media supplemented with platelet lysate (PL) or fetal calf serum (FCS). In some embodiments of the disclosure, methods of producing fibroblasts are contemplated. The starting material for the fibroblasts may be various tissues, and in one particular embodiment starting tissue is skin cells obtained from cosmetic surgery. In other embodiments, bone marrow, placenta, umbilical cord, mobilized peripheral blood, and/or omentum is isolated from healthy donors. In some cases, these donors are mammals. In specific cases, these mammals are humans. In one embodiment, dermal derived fibroblasts are cultured in tissue culture flasks between 2 and 10 days prior to washing non-adherent cells from the flask. Optionally, the number of days of culture of dermal fibroblast cells prior to washing non-adherent cells is 2 to 3 days. Preferably the fibroblasts are cultured in platelet lysate (PL) containing media. For example, 300 82 l of fibroblasts is cultured in 15 ml of PL supplemented medium in T75 or other adequate tissue culture vessels. After washing away the non-adherent cells, the adherent cells are also cultured in media that has been supplemented with platelet lysate (PL).

Thrombocytes are a well characterized human product that is widely used in clinics for patients in need of blood supplement. Thrombocytes are known to produce a wide variety of factors, e.g. PDGF-BB, TGF-β, IGF-1, and VEGF. In some embodiments, an optimized preparation of PL is used for the fibroblasts in which thrombocytes are utilized. This optimized preparation of PL is comprised of PL (including pooled platelet rich plasmas (PRPs) from at least 10 donors, such asto equalize for differences in cytokine concentrations) with thrombocytes, including with a minimal concentration of 3×10⁹ thrombocytes/ml, for example.

In particular embodiments of the disclosure, fibroblasts are cultured under conditions that allow for migration of the fibroblast to tumor cells. This may be accomplished through several mechanisms of tissue culture modification, with a goal of enhancing certain marker expression on fibroblasts, such as CXCR-4 expression on the fibroblasts. Culture conditions may include incubation under conditions of hypoxia, incubation under conditions of acidosis, incubation with histone deacetylase inhibitors, incubation with DNA methyltransferase inhibitors, and cellular fusion with cells possessing a more undifferentiated state.

Certain method of the disclosure concern culturing the fibroblast cells obtained from human tissue samples. In one embodiment, fibroblasts are plated onto a substrate. In embodiments of the present disclosure, fibroblasts are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain embodiments cells are introduced into a culture system that features at least one substrate surface that is generally compatible with adherence of cells thereto, such that the plated cells can contact the said substrate surface, such embodiments encompass plating onto a substrate, which allows adherence of cells thereto.

General principles of maintaining adherent cell cultures are well-known in the art. As appreciated by those skilled in the art, the fibroblast cells may be counted in order to facilitate subsequent plating of the cells at a desired density. Where, as in the present disclosure, the cells after plating may primarily adhere to a substrate surface present in the culture system (e.g., in a culture vessel), the plating density may be expressed as number of cells plated per mm² or cm² of the said substrate surface. In practicing the disclosure, after plating of the fibroblasts, the cell suspension may be left in contact with the adherent surface to allow for adherence of cells from the cell population to the said substrate. In contacting fibroblasts to the adherent substrate, the cells may be advantageously suspended in an environment comprising at least a medium, in the methods of the disclosure typically a liquid medium, which supports the survival and/or growth of the cells. The medium may be added to the system before, together with or after the introduction of the cells thereto. The medium may be fresh, i.e., not previously used for culturing of cells, or may comprise at least a portion which has been conditioned by prior culturing of cells therein, for example, culturing of the cells which are being plated or antecedents thereof, or culturing of cells more distantly related to or unrelated to the cells being plated.

Media used in the practice of the disclosure may be a suitable culture medium as described elsewhere in this specification. Preferably, the composition of the medium may have the same features, may be the same or substantially the same as the composition of medium used in the ensuing steps of culturing the attached cells. Otherwise, the medium may be different. The cells from the fibroblast cell population or from tissue explants of the present disclosure, which have adhered to the said substrate, preferably in the said environment, are subsequently cultured for at least 7 days, for at least 8 days, or for at least 9 days, for at least 10 days, at least 11, or at least 12 days, at least 13 days or at least 14 days, for at least 15 days, for at least 16 days or for at least 17 days, or even for at least 18 days, for at least 19 days or at least 21 days or more. The term “culturing” is common in the art and broadly refers to maintenance and/or growth of cells and/or progeny thereof.

In certain embodiments, cells of the disclosure may be fibroblasts from various tissues, selected for specific properties associated with regenerative activity. Tissues useful for the practice of the disclosure may be tissues associated with regenerative activity. Said tissues include placenta, endometrial cells, Wharton's jelly, bone marrow, and adipose tissue. In a particular embodiment, fibroblast cells are selected for expression of the markers CD117, CD105, and/or expression of the rhodamine 123 efflux activity. In some embodiments of the disclosure, fibroblasts are selected for expression of additional markers selected from the group consisting of the additional markers of Oct-4, CD-34, KLF-4, Nanog, Sox-2, Rex-1, GDF-3, Stella, possessing enhanced expression of GDF-11, and a combination thereof. Selection of fibroblasts for expression of the markers may be performed by initial expression of proteins found on the membrane of the cells, which result in possessing other markers mentioned.

### III. Modifications to Cells

The current disclosure provides methods and/or means for changing the phenotype of fibroblast and fibroblastoid-like cells. In particular embodiments, the disclosure provides means and/or methods of dedifferentiating fibroblasts to endow expression or production of one or more tumor antigens as well as one or more tumor oncogenes, without induction of full tumorigenicity. The fibroblasts may be manipulated by exposure to one or more conditions that induce dedifferentiation, in some cases. The exposure may be of any length or any intensity in order to induce dedifferentiation. By using epigenetic modifications, culture in human cord blood serum, inhibition of GSK-3, and/or other modifications, the present disclosure allows for dedifferentiation and expression of an immunological phenotype similar to that found in cancer cells, while lacking the cancer secreted immune suppressive activity.

In some embodiments, the modification of fibroblasts induces the expression or production of at least one antigen or oncogene including, but not limited to, hTERT, c-myc, k-RAS, CTCFL, AF4/HRX, ABL, ALK, AKT-2, ALK/NPM, AML1, AML1/MTG8, AXL, BCL-2, BCL-3, BCL-6, BCL-XL, BCR/ABL, DBL, DEK/CAN, E2A/PBX1, EGFR, ENL/HRX, ERG/TLS, ERBB, ERBB-2, ETS-1, EWS/FLI-1, FMS, FOS, FPS, GLI, GSP, HOX11, HER2/neu, HST, INT-2, JUN, KIT, KS3, K-SAM, LBC, LCK, LM02/LM01, LYL-1, LYT-10, TSK, TRK, Fos-related antigen 1, LCK, FAP, VEGFR2, NA17, PDGFR-beta, PAP, MAD-CT-2, Tie-2, PSA, protamine 2, legumain, endosialin, prostate stem cell antigen, carbonic anhydrase IX, STn, Page4, proteinase 3, GM3 ganglioside, tyrosinase, MART1, gp100, SART3, RGS5, SSX2, Globo11, Tn, CEA, hCG, PRAME, XAGE-1, AKAP-4, TRP-2, B7H3, sperm fibrous sheath protein, CYP1B1, HMWMAA, sLe(a), MAGE Al, GD2, PSMA, mesothelin, fucosyl GM1, GD3, sperm protein 17, NY-ESO-1, PAX5, AFP, polysialic acid, EpCAM, MAGE-A3, mutant p53, ras, mutant ras, NY-BR1, PAX3, HER2/neu, OY-TES1, HPV E6 E7, PLAC1, hTERT, BORIS, ML-IAP, idiotype of b cell lymphoma or multiple myeloma, EphA2, EGFRvIII, cyclin B 1, RhoC, androgen receptor, surviving, MYCN, wildtype p53, LMP2, ETV6-AML, MUC1, BCR-ABL, ALK, WT1, ERG (TMPRSS2 ETS fusion gene), sarcoma translocation breakpoint, STEAP, OFA/iLRP, Chondroitin sulfate proteoglycan 4 (CSPG4), alphaGal, or a combination thereof.

In some embodiments, the fibroblasts are manipulated to selectively migrate towards a tumor or cancer cells that are not in a tumor. The fibroblasts may selectively migrate towards a tumor or cancer cells because of their expression of CXCR4.

a. Expression Vectors

In embodiments wherein recombination technology is employed, one or more types of fibroblast cells are manipulated to harbor at least one expression vector that encodes at least one gene product of interest, such as a cancer antigen or multiple cancer antigens. A recombinant expression vector(s) can be introduced as one or more DNA molecules or constructs, where there may be at least one marker that will allow for selection of host cells that contain the vector(s). The vector(s) can be prepared in conventional ways, wherein the genes and regulatory regions may be isolated, as appropriate, ligated, cloned in an appropriate cloning host, and analyzed by sequencing or other convenient means. Particularly, using PCR, individual fragments including all or portions of a functional unit may be isolated, where in some cases one or more mutations may be introduced using “primer repair”, ligation, in vitro mutagenesis, etc. as appropriate. The vector(s) once completed and demonstrated to have the appropriate sequences may then be introduced into the host cell by any convenient means. The constructs may be integrated and packaged into non-replicating, defective viral genomes like lentivirus, adenovirus, adeno-associated virus (AAV), herpes simplex virus (HSV), or others, including retroviral vectors, for infection or transduction into cells. The vector(s) may include viral sequences for transfection, if desired. In some embodiments, the construct may be introduced by fusion, electroporation, biolistics, transfection, lipofection, or the like. The host cells may be grown and expanded in culture before introduction of the vector(s), followed by the appropriate treatment for introduction of the vector(s) and integration of the vector(s). The cells may then be expanded and screened by virtue of a marker present in the construct. Various markers that may be used successfully include hprt, neomycin resistance, thymidine kinase, hygromycin resistance, etc.

The vector(s) may be introduced as a single DNA molecule encoding at least one agent (including one or more angiogenic agent or functional fragments thereof) and optionally another polynucleotide (such as genes), or different DNA molecules having one or more polynucleotides (such as genes). The vector(s) may be introduced simultaneously or consecutively, each with the same or different markers. In an illustrative example, one vector would contain one or more agents (such as angiogenic agent(s)) under the control of particular regulatory sequences.

Vector(s) comprising useful elements such as bacterial or yeast origins of replication, selectable and/or amplifiable markers, promoter/enhancer elements for expression in prokaryotes or eukaryotes, etc. that may be used to prepare stocks of vector DNAs and for carrying out transfections are well known in the art, and many are commercially available.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

In some embodiments, it may be desirable to kill the modified cells, such as when the object is to terminate the treatment (for example, because of success or lack thereof), the cells become neoplastic, in research where the absence of the cells after their presence is of interest, and/or another event. For this purpose one can provide for the expression of certain gene products in which one can kill the modified cells under controlled conditions, such as a suicide gene. Suicide genes are known in the art, e.g. the iCaspase9 system in which a modified form of caspase 9 is dimerizable with a small molecule, e.g. AP1903. See, e.g., Straathof et al., Blood 105:4247-4254 (2005).

In some embodiments, the cells of the disclosure may be manipulated to express or produce one or more exogenous molecules, such as an antigen or immune stimulatory molecule. The cells may be manipulated in any method known in the art for expressing exogenous molecules including, but not limited to, via transfection, transduction, transformation, electroporation, biolistics, membrane/cell fusion, peptide pulsing, or a combination thereof. The exogenous molecule may be any molecule, including antigens or oncogenes described herein.

Expression molecules, such as expression vectors, mRNAs, or the like, may be transfected, transduced, transformed, electroporated, biolistic transformation, or otherwise introduced into a cell of the disclosure. The expression vector, mRNA, or the like may encode for one or more antigens, including antigens of the present disclosure. The expression vector, mRNA, or the like may encode the sequence for the entirety of, or any peptide derived from, a protein selected from the group consisting of hTERT, c-myc, k-RAS, CTCFL, AF4/HRX, ABL, ALK, AKT-2, ALK/NPM, AML1, AML1/MTG8, AXL, BCL-2, BCL-3, BCL-6, BCL-XL, BCR/ABL, DBL, DEK/CAN, E2A/PBX1, EGFR, ENL/HRX, ERG/TLS, ERBB, ERBB-2, ETS-1, EWS/FLI-1, FMS, FOS, FPS, GLI, GSP, HOX11, HER2/neu, HST, INT-2, JUN, KIT, KS3, K-SAM, LBC, LCK, LM02/LM01, LYL-1, LYT-10, TSK, TRK, and a combination thereof. The expression vector, mRNA, or the like may encode the sequence for the entirety of, or any peptide derived from, a protein selected from the group consisting of Fos-related antigen 1, LCK, FAP, VEGFR2, NA17, PDGFR-beta, PAP, MAD-CT-2, Tie-2, PSA, protamine 2, legumain, endosialin, prostate stem cell antigen, carbonic anhydrase IX, STn, Page4, proteinase 3, GM3 ganglioside, tyrosinase, MART1, gp100, SART3, RGS5, SSX2, Globoll, Tn, CEA, hCG, PRAME, XAGE-1, AKAP-4, TRP-2, B7H3, sperm fibrous sheath protein, CYP1B1, HMWMAA, sLe(a), MAGE Al, GD2, PSMA, mesothelin, fucosyl GM1, GD3, sperm protein 17, NY-ESO-1, PAX5, AFP, polysialic acid, EpCAM, MAGE-A3, mutant p53, ras, mutant ras, NY-BR1, PAX3, HER2/neu, OY-TES1, HPV E6 E7, PLAC1, hTERT, BORIS, ML-IAP, idiotype of b cell lymphoma or multiple myeloma, EphA2, EGFRvIII, cyclin B 1, RhoC, androgen receptor, surviving, MYCN, wildtype p53, LMP2, ETV6-AML, MUC1, BCR-ABL, ALK, WT1, ERG (TMPRSS2 ETS fusion gene), sarcoma translocation breakpoint, STEAP, OFA/iLRP, Chondroitin sulfate proteoglycan 4 (CSPG4), and a combination thereof.

The expression vector, mRNA, or the like may encode the sequence for an immune stimulatory gene. The immune stimulatory gene may be TRAIL, TNF-alpha, interferon gamma, interferon alpha, interferon beta, IL-12, IL-18, IL-21, IL-28, alpha-1,3-galactosyltransferase, or a combination thereof. The immune stimulatory molecule may induce the expression of at least one endogenous and/or exogenous antigen, including an tumor antigen and/or oncogene described herein.

For the practice of the disclosure, there are different promoters that can be used for different needs of the disclosure. It is known that different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell. As such, promoters are often classified as “constitutive”, “tissue-specific”, “cell-type-specific”, or “inducible”, depending on their functional activities in vivo or in vitro. In some situations, such as in which intratumoral injection is feasible, the use of constitutive, and maybe in some cases tissue specific, promotors are used. It is known that a constitutive promoter is one that is capable of directing transcription of a gene in a variety of cell types. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerate kinase (PGK), pyruvate kinase, phosphoglycerate mutase, the β-actin promoter, and other constitutive promoters known to those of skill in the art.

“Tissue-specific” or “cell-type-specific” promoters, on the other hand, direct transcription in some tissues and cell types but are inactive in others. Exemplary tissue-specific promoters include the PSA promoter, the probasin promoter, and the MUC1 promoter. An “inducible” promoter is one for which the transcription level of an operably linked gene varies based on the presence of a certain stimulus. Genes that are under the control of inducible promoters are expressed only, or to a greater degree, in the presence of an inducing agent, (e.g., transcription under control of the metallothionein promoter is greatly increased in presence of certain metal ions). Inducible promoters include transcriptional regulatory elements (TREs), which stimulate transcription when their inducing factors are bound. For example, there are TREs for serum factors, steroid hormones, retinoic acid and cyclic AMP. Promoters containing a particular TRE can be chosen in order to obtain an inducible response, and in some cases, the TRE itself can be attached to a different promoter, thereby conferring inducibility to the recombinant gene.

Hypoxia inducible promoter refers to a promoter that contains hypoxia responsive elements such that the active form of a hypoxia inducible factor (HIF), if present, will bind and cause the transcription of an operably linked nucleotide sequence to be enhanced above basal levels. As such, a hypoxia inducible promoter is one from which under normoxic conditions an operably linked nucleotide sequence is transcribed at basal levels or below due to the absence of an active HIF. In addition, as used herein with regard to the presently claimed subject matter, the presence of an active HIF in a cell includes not only conditions wherein the cell experiences hypoxia, but also includes any other condition where the active form of a HIF accumulates and is available to bind an HRE. Such other conditions include conditions wherein the interaction between a HIF and pVHL, and hence the ubiquitylation and degradation of the HIF, does not occur. For example, an active HIF can be formed as a result of a modification in the activity of a prolyl hydroxylase polypeptide (e.g. a mutation) such that the hydroxylation of a HIF does not occur. Alternatively, in cells that lack pVHL, active HIFs accumulate. “Normoxic conditions” or “normoxia” refer to a state of normal oxygen saturation in which HIF polypeptides are hydroxylated by prolyl hydroxylase as described above, and thus a cell does not accumulate the an active form of one or more HIFs.

The transcription of any expression vector disclosed herein may be from the presence of one or more promoters in the vector that drive the expression of one or more genes encoded by the vector. The promoter may be any promoter, including a universal promoter, house-keeping promoter, tissue-specific promoter, cell-type-specific promoter, inducible promoter, or a combination thereof. The promoter may be induced by specific signals and/or contexts. Specific signals and/or contexts that may induce a promoter include hypoxia and/or acidic conditions; compositions, including small molecules, that induce the promoter; or other tumor specific signals. The promoter may be drug-inducible. The promoter may be a hypoxia-inducible promoter, including a promoter with a hypoxia inducible factor (HIF) response element. Hypoxic conditions for driving hypoxia-inducible vectors include an oxygen tension ranging from 0.1%-10%. In some embodiments, hypoxic conditions for driving hypoxia-inducible vectors include an oxygen tension ranging from 0.1%-5%. In some embodiments, hypoxic conditions for driving hypoxia-inducible vectors include an oxygen tension less than 5%. In some embodiments, hypoxic conditions for driving hypoxia-inducible vectors include an oxygen tension of approximately 3%. The cells may be exposed to an oxygen tension disclosed herein for any period of time, including for approximately 1-60 minutes, including 1, 5, 10, 15, 20, 25, 30, 35, 40, 45, 50, 55, 60 minutes. The cells may be exposed to an oxygen tension disclosed herein for any period of time, including for approximately 1, 2, 3, 4, 5, 6, 7, 8, 9, 10, 11, 12, 13, 14, 15, 16, 17, 18, 19, 20, 21, 22, 23, 24 hours. The cells may be exposed to an oxygen tension disclosed herein for any period of time, including for approximately 1, 2, 3, 4, 5 or more days. In some embodiments, the cells may be exposed to acidic conditions, such as a pH range from approximately 5-7.4.

In some embodiments of the disclosure, stable transfection of fibroblasts is performed with the alpha(1,3)galactosyltransferase (alphaGT) gene. The gene may be placed under control of a constitutive promoter such as the CMV promoter. This will allow consistent gene expression, which may be useful for embodiments wherein the cells are used for intratumoral implantation or intratumoral injection. The reason for intratumoral implantation or intratumoral injection with cells having consistent gene expression may include if systemic administration of fibroblasts is performed in a recipient, if the fibroblasts express the alphaGT gene, the gene product, alphaGal, will cause immediate rejection and the fibroblasts will not be able to home to the tumor.

In certain embodiments, wherein systemic administration of fibroblasts expressing alphaGT is desired, using means such as intravenous administration, the alphaGT gene is made to “sleep” until it is needed for activation. Utilization of inducible promoters is known in the art and incorporated by reference. A type of inducible promoters that may be utilized in the practice of the disclosure are promoters which are activated under conditions associated with the tumor microenvironment such as conditions of hypoxia and/or acidity.

b. Signals for Antigen Expression

In some embodiments, fibroblasts are treated in combination using 5-azacytidine and/or lenolenimide in order to augment expression of tumor antigens, as well as to increase immunogenicity. One of skill in the art is referred to the publication of Sulek et al for augmentation of immunogenicity and incorporated by reference [1]. Augmentation of immunogenicity of fibroblasts expressing tumor antigens as a result of dedifferentiation may be accomplished by transfection of the fibroblasts with immune stimulatory molecules and/or cytokines. Techniques for transfection of immunogenic cytokines into mesenchymal stem cells or fibroblasts are known in the art, which under the practice of the current disclosure, may be applied to fibroblasts. Said cytokines include TRAIL [2-11], TNF-alpha [12], interferon gamma [13], interferon alpha [14], interferon beta [15-18], IL-12 [19, 20], IL-18 [21, 22], IL-21 [23], IL-28 [24],

In one embodiment, the disclosure encompasses fibroblasts that are dedifferentiated using gene transfection by introduction of one or more genes encoding proteins capable of reprogramming the fibroblasts. Protocols are known in the literature for retrodifferentiation of fibroblasts and are incorporated by reference [25-27]. Utilization of various genes, histone deacetylase inhibitors, DNA methyltransferase inhibitors, GSK-3 inhibitors, and miRNAs may be utilized to induce dedifferentiation.

In some embodiment of the present disclosure, fibroblasts are cultured under hypoxia, acidic conditions, or both. Exposure to hypoxia and/or acidic conditions may induce the expression of at least one antigen, including antigens of the present disclosure. Fibroblasts may be cultured in hypoxia, wherein the cells are exposed to less than atmospheric levels of oxygen. In some embodiments, the cells are exposed to an oxygen tension between 0.1%-10%. The cells may be exposed to an oxygen tension between 0.1%-5%. The cells may be exposed to an oxygen tension that is less than 5%. The cells may be exposed to an oxygen tension disclosed herein for any period of time, including for approximately 1-60 minutes, including 1, 5, 10, 15, 20, 25, 30, 35, 40, 45, 50, 55, 60 minutes. The cells may be exposed to an oxygen tension disclosed herein for any period of time, including for approximately 1, 2, 3, 4, 5, 6, 7, 8, 9, 10, 11, 12, 13, 14, 15, 16, 17, 18, 19, 20, 21, 22, 23, 24 hours. The cells may be exposed to an oxygen tension disclosed herein for any period of time, including for approximately 1, 2, 3, 4, 5 or more days. In some embodiments, the cells may be exposed to acidic conditions, such as a pH range from approximately 5-7.4.

In particular embodiments, a composition capable of dedifferentiating fibroblasts according to the present disclosure comprises reversin, cord blood serum, lithium, a GSK-3 inhibitor, resveratrol, pterostilbene, selenium, a selenium-containing compound, EGCG ((−)-epigallocatechin-3-gallate), valproic acid, salts of valproic acid (including sodium valproate), or a combination thereof. In some embodiments of the present disclosure, a concentration of reversin from 0.5 to 10 μM, including 1 μM is added to the fibroblast culture. Some embodiments of the present disclosure utilize resveratrol in a concentration of 10 to 100 μM, such as 50 μM. I Some embodiments of the present disclosure utilize selenium or a selenium containing compound in a concentration from 0.05 to 0.5 μM, such as 0.1 μM. In some embodiments, cord blood serum is added at a concentration of 0.1%-20% volume to the volume of tissue culture media. Some embodiments use EGCG in a concentration from 0.001 to 0.1 μM, preferably of 0.01 μM. Some embodiments use valproic acid or sodium valproate in a concentration from 1 to 10 μM, in particular of 5 μM.

In some embodiments, the fibroblasts are exposed to cytoplasmic contents of different cells, such as dedifferentiated cells and/or pluripotent cells, which may induce dedifferentiation or the expression of antigens and/or oncogenes. The cells from which the cytoplasmic content is collected may be from any source including induced pluripotent stem cells or parthenogenic derived stem cell. In some embodiments the cytoplasmic content is prepared by washing cells (i.e. the cells from which the cytoplasmic content is collected) in buffered saline, followed by lysing the cells in suitable lysis buffer. The lysed cells may be sedimented via centrifugation, resuspended in lysis buffer and incubated for sufficient time (such as 30-45 min) on ice then sonicated until all cells and nuclei are sufficiently lysed. The lysate may be sedimented by centrifugation. The resulting supernatant may be used as cytoplasmic content as described herein. Any modification to the provided preparation that produce sufficiently similar results may also be used. The fibroblasts of the present disclosure may be exposed to the cytoplasmic contents in any manner. In some embodiments, the fibroblasts are first exposed to streptolysin O (SLO), including at a concentration of approximately 230 ng/mL, then suspended in the prepared cytoplasmic content. The fibroblasts may be exposed to the cytoplasmic content for any length of time sufficient to induce dedifferentiation or expression of one or more antigens, including antigens described herein. The fibroblasts may be exposed to the cytoplasmic content for approximately 1-60 minutes, including 1, 5, 10, 15, 20, 25, 30, 35, 40, 45, 50, 55, 60 minutes, or for 1, 2, 3, 4, 5, 6, 7, 8, 9, 10, 11, 12, 13, 14, 15, 16, 17, 18, 19, 20, 21, 22, 23, 24 hours, or for 1, 2, 3, 4, 5 or more days.

IV. Tumor Antigens and Cancer Immunoregulation

Aspects of the disclosure may aim to induce immunity to tumor antigens. In previous studies immunization was performed with single antigens, such as MAGE, BAGE, and tyrosinase, in other studies, combined tumor antigens where used for immunization, wherein the combination comprised recombinant antigens. Some embodiments of the present disclosure aim to replicate tumor antigens in a tumor-like milieu, without the presence of tumor derived immune suppressive molecules. It is known that optimal T cell activation requires simultaneous signals through the T cell receptor and costimulatory molecules. The costimulatory molecule CD28, upon interaction with its ligands B7-1 and B7-2, plays a crucial role in initial T cell priming. However, the CD28-mediated T cell expansion is opposed by the B7-½ counter receptor, cytotoxic T lymphocyte associated antigen 4 (CTLA-4), which mitigates the proliferation of recently activated T cells. This sequential regulation of CD28 and CTLA-4 expression balances the activating and inhibitory signals and ensures the induction of an effective immune response, while protecting against the development of autoimmunity. Blocking of CTLA-4 with monoclonal antibodies has demonstrated some success in human clinical trials. Additional CD28 and B7 family members have been identified: PD-1 (programmed death-1), PD-L1 (programmed death ligand-1 or B7-H1), and PD-L2 (B7-DC). As in the CTLA-4/B7 system, the PD-1 interactions with PD-L1 and PD-L2 suppress both central and peripheral immune responses, and therefore, the PD-1 blockade is also being explored in clinical trials. In addition, numerous new agents targeting the inhibitory and activation pathways involved in T-cell modulation such as LAG-3, B7-H3, CD40, OX40, CD137 and others are in active development.

Accordingly, in some embodiments, T-cell induction and/or generation of an immune response in an individual as described herein comprises administration of an agonist that activates at least one co-stimulatory molecule. In some embodiments, the method comprises administration of agonistic antibodies directed against activating co-stimulatory molecules. In some embodiments, T-cell induction comprises administration of agonistic antibodies against a co-stimulatory molecule selected from the group consisting of: CD28, OX40, GITR, CD137, CD27, HVEM, and a combination thereof.

In some embodiments, T-cell induction and/or generation of an immune response in an individual as described herein comprises administration of a treatment that antagonizes negative co-stimulatory molecules. In some embodiments, the method comprises administration of blocking antibodies against negative co-stimulatory molecules. In some embodiments, T-cell induction comprises administration of blocking antibodies against a negative co-stimulatory molecule selected from the group consisting of CTLA-1; PD-1, TIM-3, BTLA, VISTA, LAG-3, and a combination thereof. In some embodiments, T-cell induction comprises administration of CTLA-4 blocking antibodies. In some embodiments, T-cell induction comprises administration of PD-1 pathway inhibitors. In some embodiments, the inhibitor of the PD-1 pathway are antibodies against PD-1 and/or soluble PD-Ll. In some embodiments, the inhibitors of the PD-1 pathway are selected from AMP-244, MEDI-4736, MPDL328 OA, and/or MIH1.

In some embodiments, T-cell induction comprises administration of a treatment that stimulates T-cell expansion. In some embodiments, a treatment that stimulates T-cell expansion comprises administration of one or more cytokines. In some embodiments, a treatment that stimulates T-cell expansion comprises administration of cytokine-expressing fibroblasts.

### V. Galactose-Alpha-1,3-Galactose

Certain aspects of the disclosure concern the treatment of cancer through manipulation of fibroblasts to either endow to the fibroblasts a constitutively immunogenic phenotype, or to manipulate fibroblasts so as to endow to them an inducible immunogenic phenotype. The immunogenic phenotype may be endowed by fibroblasts expressing one or more galactose-α-1,3-galactose (alphaGal) epitopes. Production of the alphaGal epitopes may be achieved through transfection of fibroblasts with the enzyme alpha(1,3)galactosyltransferase (alphaGT). In some embodiments, administration of fibroblasts is desired intratumorally, for example under guided imagery or where the tumor is accessible from the skin, and constitutive expression of alphaGal may be desired. In situations where the tumor is primarily accessible through circulation, inducible alphaGal is desired so as to stimulate the immune response only when the transfected fibroblasts are in the tumor and/or the tumor environment.

For the practice of the disclosure, the alphaGal epitopes (including Ga1α(1,3)Ga1β(1,4)G1cNAc-R) that are used in a constitutive or inducible manner would be recognized by natural anti-alphaGal antibodies existing in humans. The formation of immunocomplexes by anti-alphaGal antibodies and alphaGal epitopes was first observed during organ xenotransplantation. When transplanting an organ from a non-primate mammal into an Old World primate, the organ is destroyed by a hyperacute reaction within minutes of transplantation [28-30]. The hyperacute rejection of xenotransplants to higher primates is mediated by the binding of anti-alphaGal antibodies from the recipient to alphaGal epitopes expressed on the xenograft and complement activation through the classic pathway. In addition, noncomplement fixing natural anti-alphaGal antibody induces antibody dependent cell-mediated cytotoxicity (ADCC) that initiates tissue damage in xenotransplants mediated by natural killer cells. The gene encoding for alpha(1,3)-galactosyltransferase (alphaGT), which catalyzes the synthesis of alphaGal epitopes on glycoproteins and glycolipids, is inactive in humans and Old World primates but is functional in other mammals. The human immune system is continuously stimulated by intestinal and pulmonary bacterial flora to produce natural antibodies that recognize alphaGal epitopes. Anti-alphaGal constitutes approximately 1% of circulating IgG.

In some embodiments of the disclosure, administration of fibroblasts, including transfected fibroblasts, is utilized to induce antibody and complement deposition in tumors, thus increasing responsiveness to checkpoint inhibitors and other immunological and/or non-immunological therapies. In one embodiment of the disclosure, fibroblast mediated induction of alphaGal in the tumor and/or in periphery of the tumor is utilized to convert tumors from “cold” to “hot” in accordance with standard immunological definitions as described in the following publications and incorporated by reference [31-41]. For example, in one study comprehensive flow cytometric immunoprofiling on both tumor and immune cells from 51 patients with non-small cell lung cancer were obtained and this analysis was integrated with clinical and histopathologic characteristics, next-generation sequencing, mRNA expression, and PD-L1 immunohistochemistry (IHC). The cytometric profiling identified an immunologically “hot” cluster with abundant CD8⁺ T cells expressing high levels of PD-1 and TIM-3 and an immunologically “cold” cluster with lower relative abundance of CD8⁺ T cells and expression of inhibitory markers. The “hot” cluster was highly enriched for expression of genes associated with T cell trafficking and cytotoxic function and high PD-L1 expression by IHC [42]. In some embodiments, fibroblasts are transfected with at least one co-stimulatory ligand that specifically binds with at least one co-stimulatory molecule selected from the group consisting of CD27, CD28, 4-1BB, OX40, CD30, CD40, PD-1, ICOS, LFA-1, CD2, CD7, LIGHT, NKG2C, B7-H3, BTLA, Toll ligand receptor and a ligand that specifically binds with CD83. These molecules may be added to enhance immunogenicity and may be inducible and/or constitutive depending on the clinical utilization.

### VI. Administration

Embodiments of the present disclosure concern the administration of compositions of the disclosure, including the fibroblasts disclosed herein, to an individual having, or at risk of having, a cancer. An individual at risk for having cancer may be an individual at risk for having cancer when compared to the general population, and such an individual may be at risk because the individual is a smoker, has a personal or family history of cancer, has had exposure to carcinogen(s), has had excessive sun exposure, and so forth. The administration of fibroblasts, including fibroblasts that are dedifferentiated and/or express or produce at least one tumor antigen/oncogene, may cause an immune response to against the tumor antigen/oncogene. Such an immune response may induce immunity to a cancer afflicting the individual, resulting in reduction or destruction of the cancer. The fibroblasts may be administered to the individual therapeutically, prophylactically, or both. The number or amount of fibroblasts administered to the individual may be sufficient to induce an immune response. The fibroblasts may be administered systemically, peritumorally, locally to the tumor, or a combination thereof.

In some embodiments, the composition administered to the individual is co-administered with one or more compositions that induce T cell activation. The composition that induces T cell activation may be a co-stimulatory agonist, such as an agonist of CD28, OX40, GITR, CD137, CD27, HVEM, or a combination thereof. The agonist may be an agonistic antibody or small molecule. The composition that induces T cell activation may be a co-inhibitory antagonist, such as an antagonist of CTLA-4, PD-1, PD-L1, AMP-244, MEDI-4736, MPDL328 OA, MIH1 or a combination thereof. The antagonist may be an antagonistic antibody or a small molecule antagonist.

In some embodiments, the immune reaction induced by the administration of compositions disclosed herein results in tumor regression, complement activation in or near the tumor, T cell activation in or near the tumor, NK cell activation in or near the tumor, M1 cell activation in or near the tumor, M2 cell suppression in or near the tumor, suppression of angiogenesis in the tumor. In some embodiments, antibodies are generated against tumor antigens/oncogenes disclosed herein. In some embodiments, administration of the compositions disclosed herein results in antibody dependent cellular cytotoxicity

In some embodiments, cancer-targeting fibroblast immunotherapy may be performed in an amount and for a time to reduce the size of the tumor, the volume of the tumor, and/or the number of tumor cells, compared to the size, volume, and/or number of tumor cells prior to administration of oxygen or fibroblasts. In certain embodiments, adoptive immunotherapy reduces the size of the tumor, the volume of the tumor, and/or the number of tumor cells to less than 100%, to less than 95%, to less than 90%, to less than 80%, to less than 70%, to less than 60%, to less than 50%, to less than 30%, or to less than 10% of its size, volume, or cell number prior to therapy. In some embodiments, the adoptive immunotherapy reduces the growth of the tumor. In certain embodiments, the adoptive immunotherapy reduces the growth rate of the tumor by 10%, by 20%, by 30%, by 40%, by 50%, by 60%, by 70%, by 80%, by 90%, or by more than 90%, as compared to the growth rate of the tumor prior to adoptive immunotherapy. In certain embodiments, the adoptive immunotherapy increases patient survival. In some aspects, the adoptive immunotherapy increases cell death of tumor or cancer cells. The therapy may delay onset of cancer, delay metastasis of the cancer, reduce the severity or grade of the cancer, and so forth.

In some embodiments of the disclosure, induction of homeostatic expansion of T cells and/or lymphocytes in the individual may be performed, and timed with administration of fibroblasts, such as fibroblasts disclosed herein, which may be tumor targeting fibroblast therapy. Means of conditioning the patient to stimulate homeostatic expansion are known in the art. In one embodiment, prior to administration of modified fibroblast cells, the method of conditioning a patient in need of a T cell therapy comprises administering to the patient a dose of cyclophosphamide between 200 mg/m²/day and 2000 mg/m²/day and a dose of fludarabine between 20 mg/m²/day and 900 mg/m²/day. The present disclosure further provides a method of reducing endogenous lymphocytes in a patient in need of a T cell therapy comprising administering to the patient a dose of cyclophosphamide between 200 mg/m²/day and 2000 mg/m²/day and a dose of fludarabine between 20 mg/m²/day and 900 mg/m²/day. The present disclosure also provides a method of increasing a serum level of a homeostatic cytokine in a patient in need of a T cell therapy comprising administering to the patient a dose of cyclophosphamide between 200 mg/m²/day and 2000 mg/m²/day and a dose of fludarabine between 20 mg/m²/day and 900 mg/m²/day. In certain embodiments, prior to conditioning, or subsequent to conditioning, or concurrent to conditioning, cytokines associated with homeostatic expansion of T cells are administered. The homeostatic cytokines comprise interleukin 7 (IL-7), interleukin 15 (IL-15), interleukin 10 (IL-10), interleukin 5 (IL-5), gamma-induced protein 10 (IP-10), interleukin 8 (IL-8), monocyte chemotactic protein 1 (MCP-1), placental growth factor (PLGF), C-reactive protein (CRP), soluble intercellular adhesion molecule 1 (sICAM-1), soluble vascular adhesion molecule 1 (sVCAM-1), or any combination thereof, and one or more of these may be utilized.

Certain embodiments of the disclosure amplify an antigen-specific immune response following immunization with a vaccine or immunological composition, including a polyvalent vaccine in which the antigenic epitopes are used for immunization together with adjuvants such as toll like receptors (TLRs). These molecules are type 1 membrane receptors that are expressed on hematopoietic and non-hematopoietic cells. At least 11 members have been identified in the TLR family. These receptors are characterized by their capacity to recognize pathogen-associated molecular patterns (PAMP) expressed by pathogenic organisms. It has been found that triggering of TLR elicits profound inflammatory responses through enhanced cytokine production, chemokine receptor expression (CCR2, CCRS and CCR7), and costimulatory molecule expression. As such, these receptors in the innate immune systems exert control over the polarity of the ensuing acquired immune response. Among the TLRs, TLR9 has been extensively investigated for its functions in immune responses. Stimulation of the TLR9 receptor directs antigen-presenting cells (APCs) towards priming potent, T_{H1}-dominated T-cell responses, by increasing the production of pro-inflammatory cytokines and the presentation of co-stimulatory molecules to T cells. CpG oligonucleotides, ligands for TLR9, were found to be a class of potent immunostimulatory factors. CpG therapy has been tested against a wide variety of tumor models in mice, and has consistently been shown to promote tumor inhibition or regression.

In some embodiments, fibroblast therapy may be used as a means to induce shedding of tumor antigens into the environment. Dendritic cells (DCs) may be administered in order to augment the process of antigen presentation. In some embodiments, the DCs are endogenous and induced to accumulate by means of administration of one or more DC chemokines. In other embodiments, dendritic cells are generated ex vivo and administered in vivo. A discussion of dendritic cells is provided to assist one of skill in the art in practicing the disclosure. Dendritic cells (DC) possess unique morphology similar to neuronal dendrites and were originally identified based on their ability to stimulate the adaptive immune system. Of importance to the field of tumor immunotherapy, dendritic cells appear to be the only cell in the body capable of activating naïve T cells [43]. The concept of dendritic cells instructing naïve T cells to differentiate into effector or memory cells is fundamental because it places the dendritic cell as the most powerful antigen presenting cell. This implies that for immunotherapeutic purposes dendritic cells do not necessarily need to be administered at high numbers in patients.

One way in which dendritic cells have been described is as sentinels of the immune system that are patrolling the body in an immature state [44, 45]. Once DC are activated, by a stimulatory signal such as a Damage Associated Molecular Patterns (DAMPS) the DC then migrate into the draining lymph nodes through the afferent lymphatics. During the trafficking process, DC degrade ingested proteins into peptides that bind to both MHC class I molecules and MHC class II molecules. This allows the DC to: a) perform cross presentation in that they ingest exogenous antigens but present peptides in the MHC I pathway; and b) activate both CD8 (via MHC I) and CD4 (via MHC II). Interestingly, lipid antigens are processed via different pathways and are loaded onto non-classical MHC molecules of the CD1 family [46]. The possibility of utilizing DC to stimulate immunity was made into reality in animal studies that took advantage of the ability of immature DC to potently phagocytose various antigens. If the antigens possessed DAMPs, or if DAMPs were present in the environment, the DC would mature and present the antigens, resulting in stimulation of potent T cell immunity. Accordingly, in the initial studies, immature DC were incubated with various antigens, subsequent to which a maturation signal (replicating natural DAMPs) was applied and the DC were injected into animals. Thus DC were utilized as a type of “cellular adjuvant”. Indeed, it was discovered that the classical adjuvants such as Fruend's Adjuvant actually contained a high concentration of DAMPs, which resulted in the stimulation of local DC at vaccination site in vivo.

Means of using DC in the context of cancer therapy are known and provided by the following references for the specific types of cancers melanoma [47-98], soft tissue sarcoma [99], thyroid [100-102], glioma [103-124], multiple myeloma ,[125-133], lymphoma [134-136], leukemia [137-144], as well as liver [145-150], lung [151-164], ovarian [165-168], and pancreatic cancer [169-171]. Some embodiments may employ extracorporeal removal of immunological blocking factors for augmentation of existing dendritic cells to infiltrate tumors. Means of assessing dendritic cell infiltration are known in the art and described in the following examples: for gastric cancer [172-175], head and neck cancer [176-180], cervical cancer [181], breast cancer [182-184], lung cancer [185], colorectal cancer [186-188], liver cancer [189, 190], gall bladder cancer [191, 192], and pancreatic cancer [193].

In some embodiments of the disclosure, specific antigens are immunized following polyvalent immunization, said specific antigens administered in the form of DNA vaccines. Numerous publications have reported animal and clinical efficacy of DNA vaccines which are incorporated by reference [194-196]. In addition to direct DNA injection techniques, DNA vaccines can be administered by electroporation [197]. The nucleic acid compositions, including the DNA vaccine compositions, may further comprise a pharmaceutically acceptable excipient. Examples of suitable pharmaceutically acceptable excipients for nucleic acid compositions, including DNA vaccine compositions, are well known to those skilled in the art and include sugars, etc. Such excipients may be aqueous or non-aqueous solutions, suspensions, and emulsions. Examples of non-aqueous excipients include propylene glycol, polyethylene glycol, vegetable oils such as olive oil, and injectable organic esters such as ethyl oleate. Examples of aqueous excipient include water, alcoholic/aqueous solutions, emulsions or suspensions, including saline and buffered media. Suitable excipients also include agents that assist in cellular uptake of the polynucleotide molecule. Examples of such agents are (i) chemicals that modify cellular permeability, such as bupivacaine, (ii) liposomes or viral particles for encapsulation of the polynucleotide, or (iii) cationic lipids or silica, gold, or tungsten microparticles which associate themselves with the polynucleotides.

For the practice of the disclosure, the fibroblast cells may be frozen in aliquots of 10⁶-10⁸ cells in 50 mL of physiologically acceptable carrier and serum albumin (HSA). In another embodiment of the method of producing fibroblasts of the disclosure, the cells are frozen in aliquots of 10⁶-10⁸ cells per kg of subject body weight, in 50 mL of physiologically acceptable carrier and human serum albumin (HSA). In one aspect of these embodiments, when a therapeutic dose is being prepared, the appropriate number of cryovials is thawed in order to provide the appropriate number of cells for the therapeutic dose. In some cases, after DMSO is diluted from the thawed cells, the number of cryovials chosen is placed in a sterile infusion bag with 5% human serum albumin. Once in the bag, the fibroblasts do not aggregate and viability remains greater than 95% for at least 6 hours even when the fibroblasts are stored at room temperature. This provides ample time to administer the fibroblasts of the disclosure to a patient in an operating room, Optionally, the physiologically acceptable carrier is PlasmaLyte A Preferably the albumin is present at a concentration of 5% w/v. Suspending10⁶-10⁸ fibroblasts of the disclosure in greater than 40 mL of physiological carrier is critical to their biological activity. If the cells are suspended in lower volumes, the cells are prone to aggregation. Administration of aggregated fibroblasts to mammalian subjects has resulted in cardiac infarction. Thus, it is crucial that non-aggregated fibroblasts be administered according to the methods of the disclosure. The presence of albumin is also critical because it prevents aggregation of the fibroblasts and also prevents the cells from sticking to plastic containers the cells pass through when administered to subjects.

VII. Immunogenic Compositions with Other Therapies

In certain embodiments, cells of the disclosure can be used in combination with one or more other therapeutic agents including but not limited to an anti-tumor, an anti-cancer agent, and the like. The other therapy may by surgery, radiation, drug therapy, chemotherapy, hormone therapy, or a combination thereof.

In particular embodiments, the anti-tumor or anti-cancer agent is a nucleic acid molecule that encodes a protein that promotes apoptosis. In certain embodiments, the anti-tumor or anti-cancer agent is an alkylating drug, a folate antagonist, a purine antagonist, a pyrimidine antagonist, a spindle poison, a podophyllotoxin, an antibiotic, a nitrosurea, an inorganic ion, a biologic response modifier, an enzyme, or a hormone. Furthermore, in certain embodiments, the adoptive immunotherapy is combined with a second treatment that augments the immune response. The second treatment may be, for example, an adjuvant and/or a cytokine. Examples of cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); epidermal growth factor; hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-α; platelet-growth factor; transforming growth factors (TGFs) such as TGF-α and TGF-β.; insulin-like growth factor-I and -II; erythropoietin (EPO); osteoinductive factors; interferons such as interferon-α, -β and -γ colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1.alpha., IL-2, IL-3, IL-4, IL-5, IL-6, IL-7, IL-8, IL-9, IL-10, IL-11, IL-12; other polypeptide factors including LIF and kit ligand (KL); or a combination thereof. Cytokines include proteins from natural sources or from recombinant cell culture and biologically active equivalents of the native sequence cytokines.

In some embodiments of the disclosure, fibroblasts possessing or producing alphaGal are utilized to induce an initial lysis of tumor, and/or immunity towards tumor, in order to sensitize tumors to the effects of checkpoint inhibitors. Examples of such checkpoint inhibitors include: a) Inhibitors of Programmed Death 1 (PD-1, CD279), such as nivolumab (OPDIVO.RTM., BMS-936558, MDX1106, or MK-34775), and pembrolizumab (KEYTRODA.RTM., MK-3475, SCH-900475, lambrolizumab, CAS Reg. No. 1374853-91-4), as well as the PD-1 blocking agents described in U.S. Pat. Nos. 7,488,802, 7,943,743, 8,008,449, 8,168,757, 8,217, 149, WO 03042402, WO 2008156712, WO 2010089411, WO 2010036959, WO 2011066342, WO 2011159877, WO 2011082400, and WO 2011161699; b) Inhibitors of Programmed Death--Ligand 1 (PD-L1, also known as B7-H1 and CD274), including antibodies such as BMS-936559, MPDL3280A), MEDI4736, MSB0010718C, and MDX1105-01); also including: atezolizumab, durvalumab and avelumab; c) Inhibitors of CTLA-4, such as ipilimumab (YERVOY.RTM., MDX-010, BMS-734016, and MDX-101), tremelimumab, antibody clone BNI3 (Abcam), RNA inhibitors, including those described in WO 1999/032619, WO 2001/029058, U.S. 2003/0051263, U.S. 2003/0055020, U.S. 2003/0056235, U.S. 2004/265839, U.S. 2005/0100913, U.S. 2006/0024798, U.S. 2008/0050342, U.S. 2008/0081373, U.S. 2008/0248576, U.S. 2008/055443, U.S. Pat. Nos. 6,506,559, 7,282,564, 7,538,095 and 7,560,438 (each incorporated herein by reference); d) Inhibitors of PD-L2 (B7-DC, CD273), such as AMP-224 (Amplimune, Inc.) and rHIgM12B7; and e) Inhibitors of checkpoint proteins, including: LAG3, such as IMP321; TIM3 (HAVCR2); 2B4; A2aR, ID02; B7H1; B7-H3 or B7H3, such as antibody MGA271; B7H4; BTLA; CD2; CD20, such as ibritumomab tiuxetan, ofatumumab, rituximab, obinutuzumab and tositumomab; CD27, such as CDX-1127; CD28; CD30, such as brentuximab vedotin; CD33, such as gemtuzumab ozogamicin; CD40; CD52, such as alemtuzumab; CD70; CD80; CD86; CD112; CD137; CD160; CD226; CD276; DR3; OX-40 (TNFRSF.sub.4 and CD134); GALS; GITR; such as TRX518; HAVCR2; HVEM; IDO1; ICOS (inducible T cell costimulator; CD278); such as MEDI570 (Medlmmune LLC) and AMG557 (Amgen); KIR; LAIR; LIGHT; MARCO (macrophage receptor with collageneous structure); PS (phosphatidylserine); SLAM; TIGIT; VISTA; and VTCN1; or a combinations thereof.

In some embodiments, the checkpoint inhibitor is an inhibitor of a checkpoint protein selected from the group of PD-1, PD-L1, and CTLA-4. In some aspects, the checkpoint inhibitor is selected from the group of an anti-PD-1 antibody, and anti-PD-L1 antibody, and an anti-CTLA-4 antibody. In some aspects, the anti-PD-1 antibody is selected from the group of nivolumab, pembrolizumab, and lambrolizumab. In some aspects, the anti-PD-L1 antibody is selected from the group of as BMS-936559, MPDL3280A, MEDI4736, MSB0010718C, and MDX1105-01. In some aspects, the anti-PD-L1 antibody is selected from the group of durvalumab, atezolizumab, and avelumab. In some aspects, the anti-CTLA-4 antibody is selected from the group of ipilimumab and tremelimumab. In some aspects, the check point inhibitor is selected from the group consisting of nivolumab, pembrolizumab, lambrolizumab, BMS-936559, MPDL3280A, MEDI4736, MSB0010718C, MDX1105-01, durvalumab, atezolizumab, avelumab, ipilimumab, and tremelimumab. In certain embodiments, the check point inhibitor is selected from the group consisting of nivolumab, pembrolizumab, lambrolizumab, durvalumab, atezolizumab, avelumab, ipilimumab, and tremelimumab. In some embodiments, the check point inhibitor is selected from the group consisting of nivolumab, pembrolizumab, durvalumab, atezolizumab, and avelumab.

The combination of polyvalent vaccines with other cellular therapies as the initial poly-immunogenic composition is envisioned in some embodiments of the present disclosure. In one embodiment cellular lysates of tumor cells, or tumor stem cells are loaded into dendritic cells or fibroblasts. In one embodiment the disclosure provides a means of generating a population of cells with tumoricidal ability that are polyvalently reactive, to which focus is added by subsequent peptide specific vaccination. The generation of cytotoxic lymphocytes may be performed, in one embodiment by extracted 50 ml of peripheral blood from a cancer patient and peripheral blood mononuclear cells (PBMC) are isolated using the Ficoll Method. PBMC are subsequently resuspended in 10 ml AIM-V media and allowed to adhere onto a plastic surface for 2-4 hours. The adherent cells are then cultured at 37° C. in AIM-V media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on or near day 7.

In particular embodiments, DCs are used to stimulate T cell and NK cell tumoricidal activity by pulsing the cells with autologous tumor lysate. Specifically, generated DC may be further purified from culture through use of flow cytometry sorting or magnetic activated cell sorting (MACS), or any other method known in the art for purifying a specific cell population, or may be utilized as a semi-pure population. DCs pulsed with tumor lysate may be added into said patient in need of therapy with the concept of stimulating NK and T cell activity in vivo, or in other embodiments, may be incubated in vitro with a population of cells containing T cells and/or NK cells. In one embodiment, DCs are exposed to agents capable of stimulating maturation in vitro and rendering them resistant to tumor derived inhibitory compounds such as arginase byproducts.

Specific means of stimulating in vitro maturation include culturing DC or DC containing populations with a toll like receptor agonist. Another means of achieving DC maturation involves exposure of DC to TNF-alpha at a concentration of approximately 20 ng/mL. In order to activate T cells and/or NK cells in vitro, cells are cultured in media containing sufficient interferon gamma for the desired response, such as approximately 1000 IU/ml of interferon gamma. Incubation with interferon gamma may be performed for any period between 2 hours to 7 days. Preferably, incubation is performed for approximately 24 hours, after which T cells and/or NK cells are stimulated via the CD3 and/or CD28 receptors. In some embodiments, T cells and/or NK cells are stimulated by addition of antibodies capable of activating CD3 and/or CD28 receptors. In some embodiments approximately, 2 vg/m1 of anti-CD3 antibody is added, optionally, together with approximately 1 μg/ml anti-CD28. In order to promote survival of T cells and/or NK cells, as well as to stimulate proliferation, a mitogen able to affect T cells and/or NK cells may be used. In one embodiment the cytokine IL-2 is utilized. Specific concentrations of IL-2 useful for aspects of the disclosure are approximately 500 u/mL IL-2. Media containing IL-2 and antibodies may be changed every 48 hours for approximately 8-14 days.

In particular embodiments, DC are included to the T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel. The immune response of the patient treated with these cytotoxic cells is assessed utilizing a variety of antigens found in tumor endothelial cells. When cytotoxic or antibody, or antibody associated with complement fixation are recognized to be upregulated in the cancer patient, subsequent immunizations may be performed utilizing peptides to induce a focusing of the immune response.

In another embodiment, DC are generated from leukocytes of patients by leukapheresis. Numerous means of leukapheresis are known in the art. In one example, a Frenius Device (Fresenius Com.Tec) is utilized with the use of the MNC program, at approximately 1500 rpm, and with a P1Y kit. The plasma pump flow rates are adjusted to approximately 50 mL/min. Various anticoagulants may be used, for example ACD-A. The Inlet/ACD Ratio may be ranged from approximately 10:1 to 16:1. In one embodiment approximately 150 mL of blood is processed. The leukapheresis product is subsequently used for initiation of dendritic cell culture. In order to generate peripheral blood mononuclear cells from leukapheresis product, mononuclear cells are isolated by the Ficoll-Hypaque density gradient centrifugation. Monocytes are then enriched by the Percoll hyperosmotic density gradient centrifugation followed by two hours of adherence to the plate culture. Cells are then centrifuged at approximately 500 g to separate the different cell populations. Adherent monocytes are cultured for approximately 7 days in 6-well plates suitable confluence, such as 2×10⁶ cells/mL. The cells may be cultured in RMPI medium with approximately 1% penicillin/streptomycin, approximately 2 mM L-glutamine, approximately 10% of autologous 50 ng/mL GM-CSF and approximately 30 ng/mL IL-4. On approximately day 6 immature dendritic cells are pulsed with fibroblast dedifferentiate cell lysate or exosomes. Pulsing may be performed by incubation of lysates with dendritic cells, or may be generated by fusion of immature dendritic cells with dedifferentiated fibroblast cells.

Means of generating hybridomas or cellular fusion products are known in the art and include electrical pulse mediated fusion, or stimulation of cellular fusion by treatment with polyethelyne glycol. On approximately day 7, the immature DCs are then induced to differentiate into mature DCs by culturing for 48 hours with 30 ng/mL interferon gamma (IFN-γ). During the course of generating DC for clinical purposes, microbiologic monitoring tests are performed at the beginning of the culture, on approximately the fifth day and at the time of cell freezing for further use or prior to release of the dendritic cells. Administration of dedifferentiated fibroblast lysate pulsed dendritic cells is utilized as a polyvalent vaccine, whereas subsequent to administration antibody or T cell responses are assessed for induction of antigen specificity, peptides corresponding to immune response stimulated are used for further immunization to focus the immune response.

In some embodiments, culture of the immune effectors cells is performed after extracting from a patient that has been immunized with a polyvalent antigenic preparation. Specifically separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4. Separation methods that depend on such surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized (for example, CD8 MicroBeads), Dynabeads M450 CD8, and Eligix anti-CD8 mAb coated nickel particles, or any other beads/particles known in the art can be suitably used. Similar implementations using CD4 as a purifying marker may also be used, for example, CD4 MicroBeads, Dynabeads M-450 CD4, or any other beads/particles known in the art can also be used.

In some embodiments of the disclosure, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-.gamma., transforming growth factor (TGF)-beta, IL-15, IL-7, IFN-alpha, IL-12, CD4OL, and IL-27. From the viewpoint of enhancing cellular immunity, particularly suitably, IL-2, IFN-.gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule.

Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current disclosure to optimize the cellular product based on other means of assessing T cell activity, for example, the function enhancement of the T cell in the method of the present disclosure can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the disclosure may be assessed in a living body before first administration of the T cell with enhanced function of the present disclosure, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide.

Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects. Said cells can be expanded in the presence of specific antigens associated with tumor endothelium and subsequently injected into the patient in need of treatment. Expansion with specific antigens includes coculture with proteins selected from a group comprising of : a) ROBO; b) VEGF-R2; c) FGF-R; d) CD105; e) TEM-1; and f) survivin. Other tumor endothelial specific or semi-specific antigens are known in the art that may be used.

### EXAMPLES

The following example is included to demonstrate certain non-limiting aspects of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1

### Inhibition of Tumor Growth Using Dedifferentiated Fibroblasts

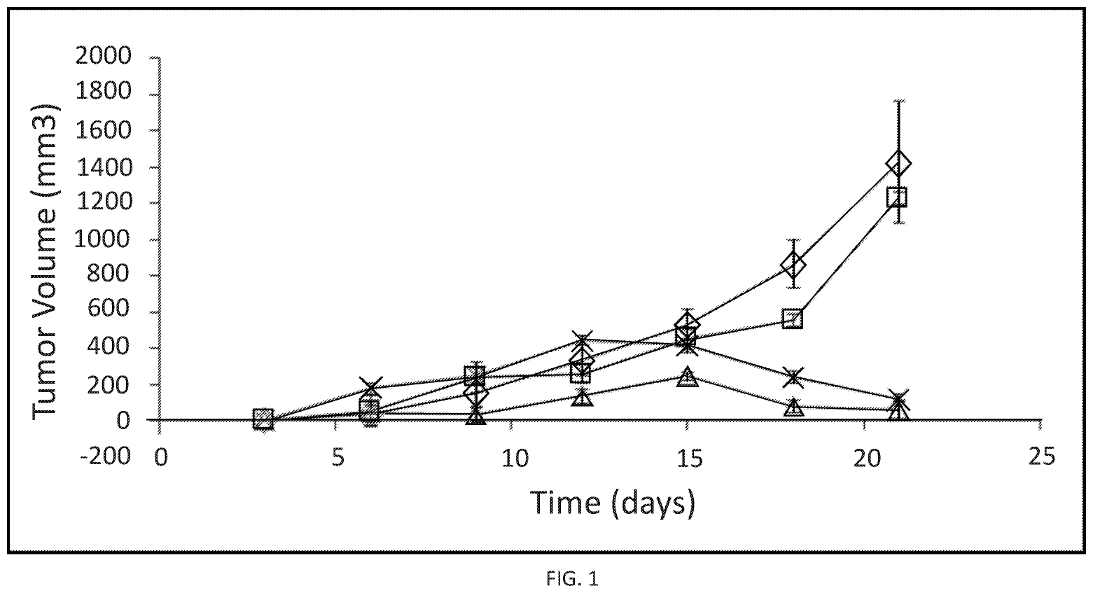

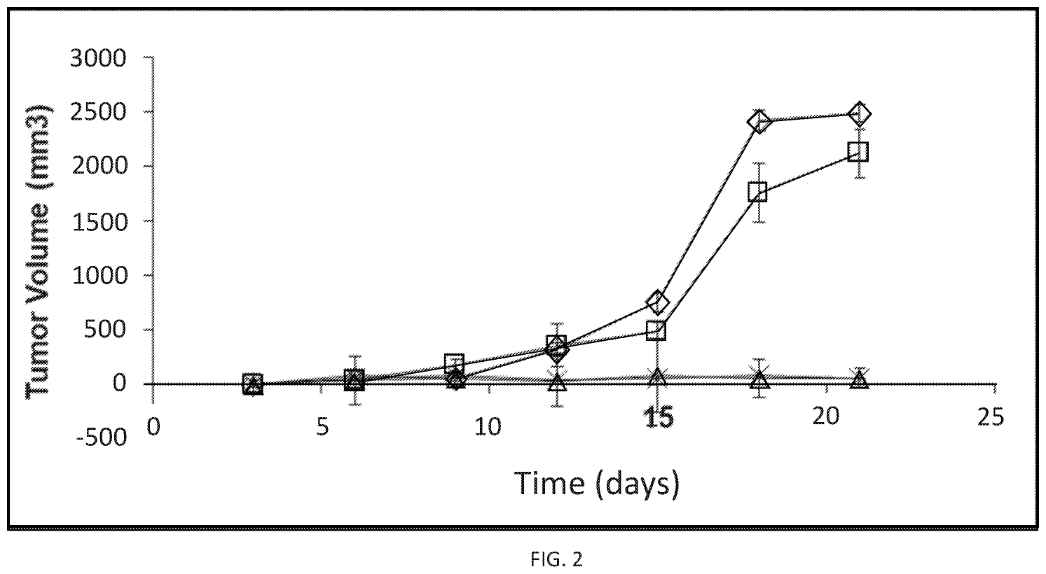

Human fibroblasts were grown in 5 azacytidine (Protocol 1) or Valproic Acid (Protocol 2) in the presence of human inducible pluripotent stem cell cytoplasm, which was administered through semi-permeabilization of membranes with streptolysin 0 according to the protocol of Collas. Dedifferentiated human fibroblasts generated with two methodologies (Protocol 1) and (Protocol 2), as well as control fibroblasts were mitotically inactivated and administered 3 days after tumor inoculation. Tumor inoculation was performed in the flank of syngeneic mice using the B16 melanoma (FIG. 1) cells and the 4T1 breast cancer (FIG. 2) cells. Inhibition of tumor growth was observed with both protocols of dedifferentiated fibroblasts but not control human fibroblasts.

### Example 2

### Dedifferentiaton of Fibroblasts Resulting in Expression of Tumor Antigen by DNA Methyltransferase Inhibition and Gsk-3 Inhibition

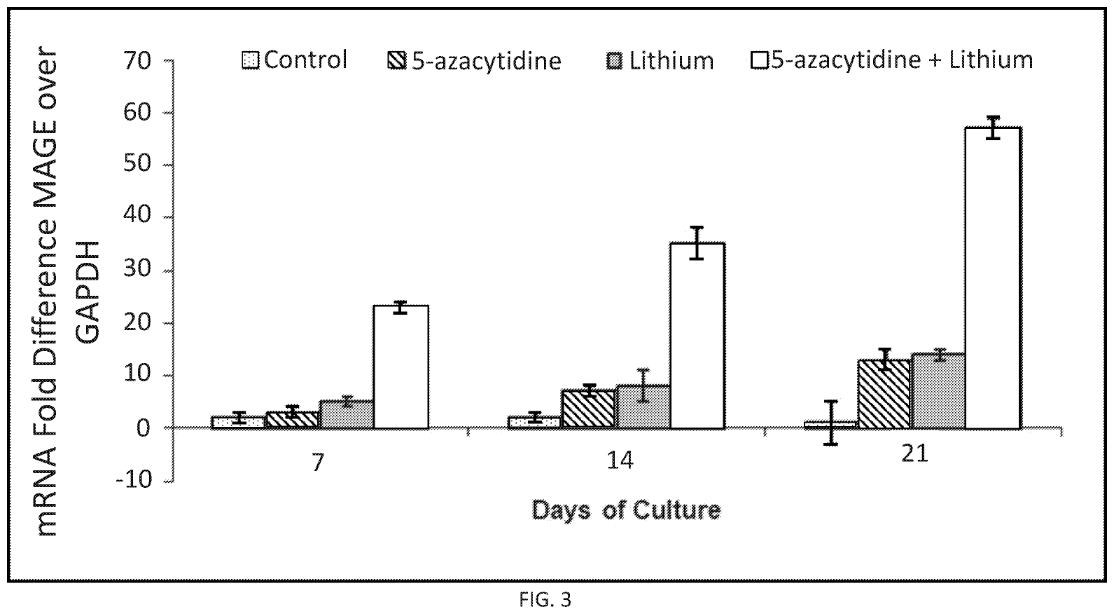

Foreskin fibroblasts were obtained from American Type Culture Collection (ATCC) and cultured according to manufacturer's instructions in Opti-MEM media with 10% fetal calf serum. Once a stable growth rate was obtained, cells were treated with control media, 5-azacytidine at a concentration of 1 microgram per ml (DNA methyltransferase inhibitor), Lithium at a concentration of 0.2 microgram per ml (GSK-3 inhibitor) and cultured for 7, 14, and 21 days. Assessment of MAGE1 gene expression was performed by PCR and expressed as % compared to GAPDH housekeeping gene (FIG. 3). Quantitative PCR was performed using a Stratagene Mx3000P instrument and the data was analyzed with MxPro software. Individual reactions contained 10 82 l Brilliant SYBR Green Master Mix (Stratagene), 2 μl cDNA (equivalent to 16 ng total RNA), oligonucleotides at a final concentration of 500 nM and H₂O for a total volume of 20 μl. The thermo cycles were as follows: 1 cycle of 10 min at 95° C.; 40 cycles of 30 s at 95° C., 60 s at 56° C., 30 s at 72° C., and 1 dissociation cycle of 30 s at 95° C., 30 s at 55° C., slow ramp to 95° C. All QPCR runs included no reverse transcriptase control reactions and a dissociation analysis of the final products in order to confirm the formation of specific PCR products from mRNA Primers used for amplification of MAGE 1 were as follows:

(SEQ ID NO: 1)
AGTAGTAGGTTTCTGTTCTATTGGG
and
(SEQ ID NO: 2)
TACTTATTCCACTGCTGTTATTATCC.

**********************************************************

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations. All references cited herein are all incorporated by reference herein, in their entirety, whether specifically incorporated or not. All publications, patents, and patent applications, cited herein are hereby expressly incorporated by reference for all purposes. In particular, all nucleotide sequences, amino acid sequences, nucleic constructs, DNA vaccines, oncolytic viruses, methods of administration, particular orders of administration of DNA vaccines and agents, such as oncolytic viruses and cell therapies that are described in the patents, patent applications and other publications referred to herein or authored by one or more of the inventors of this application are specifically incorporated by reference herein. In case of conflict, the definitions within the instant application govern.

The scope of the present application is not intended to be limited to the particular embodiments of the process, machine, manufacture, composition of matter, means, methods and steps described in the specification.

### REFERENCES

1. Sulek, J. E., et al., Role of Epigenetic Modification and Immunomodulation in a Murine Prostate Cancer Model. Prostate, 2017. 77(4): p. 361-373.

2. Loebinger, M. R., et al., Mesenchymal stem cell delivery of TRAIL can eliminate metastatic cancer. Cancer Res, 2009. 69(10): p. 4134-42.

3. Xia, L., et al., TRAIL-expressing gingival-derived mesenchymal stem cells inhibit tumorigenesis of tongue squamous cell carcinoma. J Dent Res, 2015. 94(1): p. 219-28.

4. Attar, R., et al., TRAIL based therapy: overview of mesenchymal stem cell based delivery and miRNA controlled expression of TRAIL. Asian Pac J Cancer Prey, 2014. 15(16): p. 6495-7.

5. Loebinger, M. R., et al., TRAIL-expressing mesenchymal stem cells kill the putative cancer stem cell population. Br J Cancer, 2010. 103(11): p. 1692-7.

6. Yuan, Z., et al., Mesenchymal stromal cell delivery of full-length tumor necrosis factor-related apoptosis-inducing ligand is superior to soluble type for cancer therapy. Cytotherapy, 2015. 17(7): p. 885-96.

7. Choi, S. A., et al., Preclinical Biosafety Evaluation of Genetically Modified Human Adipose Tissue-Derived Mesenchymal Stem Cells for Clinical Applications to Brainstem Glioma. Stem Cells Dev, 2016. 25(12): p. 897-908.

8. Yan, C., et al., Human umbilical cord mesenchymal stem cells delivering sTRAIL home to lung cancer mediated by MCP-1/CCR2 axis and exhibit antitumor effects. Tumour Biol, 2016. 37(6): p. 8425-35.

9. Grisendi, G., et al., Mesenchymal progenitors expressing TRAIL induce apoptosis in sarcomas. Stem Cells, 2015. 33(3): p. 859-69.

10. Zhang, B., et al., The inhibitory effect of MSCs expressing TRAIL as a cellular delivery vehicle in combination with cisplatin on hepatocellular carcinoma. Cancer Biol Ther, 2012. 13(12): p. 1175-84.

11. Ciavarella, S., et al., In vitro anti-myeloma activity of TRAIL-expressing adipose-derived mesenchymal stem cells. Br J Haematol, 2012. 157(5): p. 586-98.

12. Zhang, X., et al., Mesenchymal stem cells modified to express lentivirus TNF-alpha Tumstatin(45-132) inhibit the growth of prostate cancer. J Cell Mol Med, 2011. 15(2): p. 433-44.

13. Yang, X., et al., IFN-gamma-secreting-mesenchymal stem cells exert an antitumor effect in vivo via the TRAIL pathway. J Immunol Res, 2014. 2014: p. 318098.

14. Su, Y., et al., Interferon-alpha2b gene-modified human bone marrow mesenchymal stem cells inhibit hepatocellular carcinoma by reducing the Notch1 levels. Life Sci, 2015. 143: p. 18-26.

15. Xie, C., et al., Interferon-beta gene-modified human bone marrow mesenchymal stem cells attenuate hepatocellular carcinoma through inhibiting AKT/FOXO3a pathway. Br J Cancer, 2013. 109(5): p. 1198-205.

16. Dembinski, J. L., et al., Tumor stroma engraftment of gene-modified mesenchymal stem cells as anti-tumor therapy against ovarian cancer. Cytotherapy, 2013. 15(1): p. 20-32.

17. Wang, G. X., et al., Mesenchymal stem cells modified to express interferon-beta inhibit the growth of prostate cancer in a mouse model. J Int Med Res, 2012. 40(1): p. 317-27.

18. Ren, C., et al., Cancer gene therapy using mesenchymal stem cells expressing interferon-beta in a mouse prostate cancer lung metastasis model. Gene Ther, 2008. 15(21): p. 1446-53.

19. Ryu, C. H., et al., Gene therapy of intracranial glioma using interleukin 12-secreting human umbilical cord blood-derived mesenchymal stem cells. Hum Gene Ther, 2011. 22(6): p. 733-43.

20. Seo, S. H., et al., The effects of mesenchymal stem cells injected via different routes on modified IL-12-mediated antitumor activity. Gene Ther, 2011. 18(5): p. 488-95.

21. Liu, X., et al., Mesenchymal stem cells expressing interleukin-18 suppress breast cancer cells in vitro. Exp Ther Med, 2015. 9(4): p. 1192-1200.

22. Sun, S., et al., [Effect of interleukin-18 gene modified human umbilical cord mesenchymal stem cells on proliferation of breast cancer cell]. Zhonghua Yi Xue Za Zhi, 2014. 94(26): p. 2013-7.

23. Kim, N., et al., IL-21-Expressing Mesenchymal Stem Cells Prevent Lethal B-Cell Lymphoma Through Efficient Delivery of IL-21, Which Redirects the Immune System to Target the Tumor. Stem Cells Dev, 2015. 24(23): p. 2808-21.

24. Suzuki, T., et al., Mesenchymal stem cells are efficiently transduced with adenoviruses bearing type 35-derived fibers and the transduced cells with the IL-28A gene produces cytotoxicity to lung carcinoma cells co-cultured. BMC Cancer, 2014. 14: p. 713.

25. Wang, Y., et al., Unique molecular events during reprogramming of human somatic cells to induced pluripotent stem cells (iPSCs) at naive state. Elife, 2018. 7.

26. Wattanapanitch, M., et al., Dual small-molecule targeting of SMAD signaling stimulates human induced pluripotent stem cells toward neural lineages. PLoS One, 2014. 9(9): p. e106952.

27. Zhang, Z. and W. S. Wu, Sodium butyrate promotes generation of human induced pluripotent stem cells through induction of the miR302/367 cluster. Stem Cells Dev, 2013. 22(16): p. 2268-77.

28. Zhong, R., et al., Improvement in human decay accelerating factor transgenic porcine kidney xenograft rejection with intravenous administration of gas914, a polymeric form of alphaGAL. Transplantation, 2003. 75(1): p. 10-9.

29. Koren, E., et al., Monoclonal antiidiotypic antibodies neutralize cytotoxic effects of anti-alphaGal antibodies. Transplantation, 1996. 62(6): p. 837-43.

30. Neethling, F. A. and D. K. Cooper, Serum cytotoxicity to pig cells and anti-alphaGal antibody level and specificity in humans and baboons. Transplantation, 1999. 67(5): p. 658-65.

31. Canton, D. A., et al., Melanoma treatment with intratumoral electroporation of tavokinogene telseplasmid (pIL-12, tavokinogene telseplasmid). Immunotherapy, 2017. 9(16): p. 1309-1321.

32. Kuczynski, E. A., et al., Impact of Chemical-Induced Mutational Load Increase on Immune Checkpoint Therapy in Poorly Responsive Murine Tumors. Mol Cancer Ther, 2018. 17(4): p. 869-882.

33. Vonderheide, R. H., The Immune Revolution: A Case for Priming, Not Checkpoint. Cancer Cell, 2018. 33(4): p. 563-569.

34. Domagala-Kulawik, J., Immune checkpoint inhibitors in non-small cell lung cancer—towards daily practice. Adv Respir Med, 2018. 86(3).

35. Collins, J. M., J. M. Redman, and J. L. Gulley, Combining vaccines and immune checkpoint inhibitors to prime, expand, and facilitate effective tumor immunotherapy. Expert Rev Vaccines, 2018. 17(8): p. 697-705.

36. Patel, R. B., et al., Combining brachytherapy and immunotherapy to achieve in situ tumor vaccination: A review of cooperative mechanisms and clinical opportunities. Brachytherapy, 2018. 17(6): p. 995-1003.

37. Gujar, S., J. G. Pol, and G. Kroemer, Heating it up: Oncolytic viruses make tumors ‘hot’ and suitable for checkpoint blockade immunotherapies. Oncoimmunology, 2018. 7(8): p. e1442169.

38. Rodallec, A., et al., Turning cold tumors into hot tumors: harnessing the potential of tumor immunity using nanoparticles. Expert Opin Drug Metab Toxicol, 2018. 14(11): p. 1139-1147.

39. LaRocca, C. J. and S. G. Warner, Oncolytic viruses and checkpoint inhibitors: combination therapy in clinical trials. Clin Transl Med, 2018. 7(1): p. 35.

40. Maleki Vareki, S., High and low mutational burden tumors versus immunologically hot and cold tumors and response to immune checkpoint inhibitors. J Immunother Cancer, 2018. 6(1): p. 157.

41. Galon, J. and D. Bruni, Approaches to treat immune hot, altered and cold tumours with combination immunotherapies. Nat Rev Drug Discov, 2019. 18(3): p. 197-218.

42. Lizotte, P. H., et al., Multiparametric profiling of non-small-cell lung cancers reveals distinct immunophenotypes. JCI Insight, 2016. 1(14): p. e89014.

43. Steinman, R. M. and Z. A. Cohn, Identification of a novel cell type in peripheral lymphoid organs of mice. I. Morphology, quantitation, tissue distribution. J Exp Med, 1973. 137(5): p. 1142-62.

44. Banchereau, J. and R. M. Steinman, Dendritic cells and the control of immunity. Nature, 1998. 392(6673): p. 245-52.

45. Trombetta, E. S. and I. Mellman, Cell biology of antigen processing in vitro and in vivo. Annu Rev Immunol, 2005. 23: p. 975-1028.

46. Itano, A. A. and M. K. Jenkins, Antigen presentation to naive CD4 T cells in the lymph node. Nat Immunol, 2003. 4(8): p. 733-9.

47. Nestle, F. O., et al., Vaccination of melanoma patients with peptide- or tumor lysate pulsed dendritic cells. Nat Med, 1998. 4(3): p. 328-32.

48. Chakraborty, N. G., et al., Immunization with a tumor-cell-lysate-loaded autologous-antigen-presenting-cell-based vaccine in melanoma. Cancer Immunol Immunother, 1998. 47(1): p. 58-64.

49. Wang, F., et al., Phase I trial of a MART-1 peptide vaccine with incomplete Freund's adjuvant for resected high-risk melanoma. Clin Cancer Res, 1999. 5(10): p. 2756-65.

50. Thurner, B., et al., Vaccination with mage-3A1 peptide pulsed mature, monocyte-derived dendritic cells expands specific cytotoxic T cells and induces regression of some metastases in advanced stage IV melanoma. J Exp Med, 1999. 190(11): p. 1669-78.

51. Thomas, R., et al., Immature human monocyte-derived dendritic cells migrate rapidly to draining lymph nodes after intradermal injection for melanoma immunotherapy. Melanoma Res, 1999. 9(5): p. 474-81.

52. Mackensen, A., et al., Phase I study in melanoma patients of a vaccine with peptide-pulsed dendritic cells generated in vitro from CD34(+) hematopoietic progenitor cells. Int J Cancer, 2000. 86(3): p. 385-92.

53. Panelli, M. C., et al., Phase 1 study in patients with metastatic melanoma of immunization with dendritic cells presenting epitopes derived from the melanoma-associated antigens MART-1 and gp100. J Immunother, 2000. 23(4): p. 487-98.

54. Schuler-Thurner, B., et al., Mage-3 and influenza-matrix peptide-specific cytotoxic T cells are inducible in terminal stage HIA-A2.1+melanoma patients by mature monocyte-derived dendritic cells. J Immunol, 2000. 165(6): p. 3492-6.

55. Lau, R., et al., Phase I trial of intravenous peptide-pulsed dendritic cells in patients with metastatic melanoma. J Immunother, 2001. 24(1): p. 66-78.

56. Banchereau, J., et al., Immune and clinical responses in patients with metastatic melanoma to CD34(+) progenitor-derived dendritic cell vaccine. Cancer Res, 2001. 61(17): p. 6451-8.

57. Schuler-Thurner, B., et al., Rapid induction of tumor-specific type 1 T helper cells in metastatic melanoma patients by vaccination with mature, cryopreserved, peptide-loaded monocyte-derived dendritic cells. J Exp Med, 2002. 195(10): p. 1279-88.

58. Palucka, A. K., et al., Single injection of CD34+progenitor-derived dendritic cell vaccine can lead to induction of T-cell immunity in patients with stage IV melanoma. J Immunother, 2003. 26(5): p. 432-9.

59. Bedrosian, I., et al., Intranodal administration of peptide pulsed mature dendritic cell vaccines results in superior CD8+T-cell function in melanoma patients. J Clin Oncol, 2003. 21(20): p. 3826-35.

60. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21(21): p. 4016-26.

61. Hersey, P., et al., Phase I/II study of treatment with dendritic cell vaccines in patients with disseminated melanoma. Cancer Immunol Immunother, 2004. 53(2): p. 125-34.

62. Vilella, R., et al., Pilot study of treatment of biochemotherapy-refractory stage IV melanoma patients with autologous dendritic cells pulsed with a heterologous melanoma cell line lysate. Cancer Immunol Immunother, 2004. 53(7): p. 651-8.

63. Palucka, A. K., et al., Spontaneous proliferation and type 2 cytokine secretion by CD4+T cells in patients with metastatic melanoma vaccinated with antigen-pulsed dendritic cells. J Clin Immunol, 2005. 25(3): p. 288-95.

64. Banchereau, J., et al., Immune and clinical outcomes in patients with stage IV melanoma vaccinated with peptide pulsed dendritic cells derived from CD34+ progenitors and activated with type I interferon. J Immunother, 2005. 28(5): p. 505-16.

65. Trakatelli, M., et al., A new dendritic cell vaccine generated with interleukin-3 and interferon-beta induces CD8+T cell responses against NA17-A2 tumor peptide in melanoma patients. Cancer Immunol Immunother, 2006. 55(4): p. 469-74.

66. Salcedo, M., et al., Vaccination of melanoma patients using dendritic cells loaded with an allogeneic tumor cell lysate. Cancer Immunol Immunother, 2006. 55(7): p. 819-29.

67. Linette, G. P., et al., Immunization using autologous dendritic cells pulsed with the melanoma-associated antigen gp100-derived G280-9V peptide elicits CD8+ immunity. Clin Cancer Res, 2005. 11(21): p. 7692-9.

68. Escobar, A., et al., Dendritic cell immunizations alone or combined with low doses of interleukin-2 induce specific immune responses in melanoma patients. Clin Exp Immunol, 2005. 142(3): p. 555-68.

69. Tuettenberg, A., et al., Induction of strong and persistent MelanA/MART-1-specific immune responses by adjuvant dendritic cell-based vaccination of stage II melanoma patients. Int J Cancer, 2006. 118(10): p. 2617-27.

70. Schadendorf, D., et al., Dacarbazine (DTIC) versus vaccination with autologous peptide-pulsed dendritic cells (DC) in first-line treatment of patients with metastatic melanoma: a randomized phase III trial of the DC study group of the DeCOG. Ann Oncol, 2006. 17(4): p. 563-70.

71. Di Pucchio, T., et al., Immunization of stage IV melanoma patients with Melan-A/MART-1 and gp100 peptides plus IFN-alpha results in the activation of specific CD8(+) T cells and monocyte/dendritic cell precursors. Cancer Res, 2006. 66(9): p. 4943-51.

72. Nakai, N., et al., Vaccination of Japanese patients with advanced melanoma with peptide, tumor lysate or both peptide and tumor lysate pulsed mature, monocyte-derived dendritic cells. J Dermatol, 2006. 33(7): p. 462-72.

73. Palucka, A. K., et al., Dendritic cells loaded with killed allogeneic melanoma cells can induce objective clinical responses and MART-1 specific CD8+T-cell immunity. J Immunother, 2006. 29(5): p. 545-57.

74. Lesimple, T., et al., Immunologic and clinical effects of injecting mature peptide-loaded dendritic cells by intralymphatic and intranodal routes in metastatic melanoma patients. Clin Cancer Res, 2006. 12(24): p. 7380-8.

75. Guo, J., et al., Intratumoral injection of dendritic cells in combination with local hyperthermia induces systemic antitumor effect in patients with advanced melanoma. Int J Cancer, 2007. 120(11): p. 2418-25.

76. O'Rourke, M. G., et al., Dendritic cell immunotherapy for stage IV melanoma. Melanoma Res, 2007. 17(5): p. 316-22.

77. Bercovici, N., et al., Analysis and characterization of antitumor T-cell response after administration of dendritic cells loaded with allogeneic tumor lysate to metastatic melanoma patients. J Immunother, 2008. 31(1): p. 101-12.

78. Hersey, P., et al., Phase I/II study of treatment with matured dendritic cells with or without low dose IL-2 in patients with disseminated melanoma. Cancer Immunol Immunother, 2008. 57(7): p. 1039-51.

79. von Euw, E. M., et al., A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. J Transl Med, 2008. 6: p. 6.

80. Carrasco, J., et al., Vaccination of a melanoma patient with mature dendritic cells pulsed with MAGE-3 peptides triggers the activity of nonvaccine anti-tumor cells. J Immunol, 2008. 180(5): p. 3585-93.

81. Redman, B. G., et al., Phase lb trial assessing autologous, tumor-pulsed dendritic cells as a vaccine administered with or without IL-2 in patients with metastatic melanoma. J Immunother, 2008. 31(6): p. 591-8.

82. Daud, A. I., et al., Phenotypic and functional analysis of dendritic cells and clinical outcome in patients with high-risk melanoma treated with adjuvant granulocyte macrophage colony-stimulating factor. J Clin Oncol, 2008. 26(19): p. 3235-41.

83. Engell-Noerregaard, L., et al., Review of clinical studies on dendritic cell-based vaccination of patients with malignant melanoma: assessment of correlation between clinical response and vaccine parameters. Cancer Immunol Immunother, 2009. 58(1): p. 1-14.

84. Nakai, N., et al., Immunohistological analysis of peptide-induced delayed-type hypersensitivity in advanced melanoma patients treated with melanoma antigen-pulsed mature monocyte-derived dendritic cell vaccination. J Dermatol Sci, 2009. 53(1): p. 40-7.

85. Dillman, R. O., et al., Phase II trial of dendritic cells loaded with antigens from self-renewing, proliferating autologous tumor cells as patient-specific antitumor vaccines in patients with metastatic melanoma: final report. Cancer Biother Radiopharm, 2009. 24(3): p. 311-9.

86. Chang, J. W., et al., Immunotherapy with dendritic cells pulsed by autologous dactinomycin-induced melanoma apoptotic bodies for patients with malignant melanoma. Melanoma Res, 2009. 19(5): p. 309-15.

87. Trepiakas, R., et al., Vaccination with autologous dendritic cells pulsed with multiple tumor antigens for treatment of patients with malignant melanoma: results from a phase I/II trial. Cytotherapy, 2010. 12(6): p. 721-34.

88. Jacobs, J. F., et al., Dendritic cell vaccination in combination with anti-CD25 monoclonal antibody treatment: a phase I/II study in metastatic melanoma patients. Clin Cancer Res, 2010. 16(20): p. 5067-78.

89. Ribas, A., et al., Multicenter phase II study of matured dendritic cells pulsed with melanoma cell line lysates in patients with advanced melanoma. J Transl Med, 2010. 8: p. 89.

90. Ridolfi, L., et al., Unexpected high response rate to traditional therapy after dendritic cell-based vaccine in advanced melanoma: update of clinical outcome and subgroup analysis. Clin Dev Immunol, 2010. 2010: p. 504979.

91. Cornforth, A. N., et al., Resistance to the proapoptotic effects of interferon-gamma on melanoma cells used in patient-specific dendritic cell immunotherapy is associated with improved overall survival. Cancer Immunol Immunother, 2011. 60(1): p. 123-31.

92. Lesterhuis, W. J., et al., Wild-type and modified gp100 peptide-pulsed dendritic cell vaccination of advanced melanoma patients can lead to long-term clinical responses independent of the peptide used. Cancer Immunol Immunother, 2011. 60(2): p. 249-60.

93. Bjoern, J., et al., Changes in peripheral blood level of regulatory T cells in patients with malignant melanoma during treatment with dendritic cell vaccination and low-dose IL-2. Scand J Immunol, 2011. 73(3): p. 222-33.

94. Steele, J. C., et al., Phase I/II trial of a dendritic cell vaccine transfected with DNA encoding melan A and gp100 for patients with metastatic melanoma. Gene Ther, 2011. 18(6): p. 584-93.

95. Kim, D. S., et al., Immunotherapy of malignant melanoma with tumor lysate pulsed autologous monocyte-derived dendritic cells. Yonsei Med J, 2011. 52(6): p. 990-8.

96. Ellebaek, E., et al., Metastatic melanoma patients treated with dendritic cell vaccination, Interleukin-2 and metronomic cyclophosphamide: results from a phase II trial. Cancer Immunol Immunother, 2012. 61(10): p. 1791-804.

97. Dillman, R. O., et al., Tumor stem cell antigens as consolidative active specific immunotherapy: a randomized phase II trial of dendritic cells versus tumor cells in patients with metastatic melanoma. J Immunother, 2012. 35(8): p. 641-9.

98. Dannull, J., et al., Melanoma immunotherapy using mature DCs expressing the constitutive proteasome. J Clin Invest, 2013. 123(7): p. 3135-45.

99. Finkelstein, S. E., et al., Combination of external beam radiotherapy (EBRT) with intratumoral injection of dendritic cells as neo-adjuvant treatment of high-risk soft tissue sarcoma patients. Int J Radiat Oncol Biol Phys, 2012. 82(2): p. 924-32.

100. Stift, A., et al., Dendritic cell vaccination in medullary thyroid carcinoma. Clin Cancer Res, 2004. 10(9): p. 2944-53.

101. Kuwabara, K., et al., Results of a phase I clinical study using dendritic cell vaccinations for thyroid cancer. Thyroid, 2007. 17(1): p. 53-8.

102. Bachleitner-Hofmann, T., et al., Pilot trial of autologous dendritic cells loaded with tumor lysate(s) from allogeneic tumor cell lines in patients with metastatic medullary thyroid carcinoma. Oncol Rep, 2009. 21(6): p. 1585-92.

103. Yu, J. S., et al., Vaccination of malignant glioma patients with peptide-pulsed dendritic cells elicits systemic cytotoxicity and intracranial T-cell infiltration. Cancer Res, 2001. 61(3): p. 842-7.

104. Yamanaka, R., et al., Vaccination of recurrent glioma patients with tumour lysate pulsed dendritic cells elicits immune responses: results of a clinical phase I/II trial. Br J Cancer, 2003. 89(7): p. 1172-9.

105. Yu, J. S., et al., Vaccination with tumor lysate pulsed dendritic cells elicits antigen-specific, cytotoxic T-cells in patients with malignant glioma. Cancer Res, 2004. 64(14): p. 4973-9.

106. Yamanaka, R., et al., Tumor lysate and IL-18 loaded dendritic cells elicits Th1 response, tumor-specific CD8+ cytotoxic T cells in patients with malignant glioma. J Neurooncol, 2005. 72(2): p. 107-13.

107. Yamanaka, R., et al., Clinical evaluation of dendritic cell vaccination for patients with recurrent glioma: results of a clinical phase I/II trial. Clin Cancer Res, 2005. 11(11): p. 4160-7.

108. Liau, L. M., et al., Dendritic cell vaccination in glioblastoma patients induces systemic and intracranial T-cell responses modulated by the local central nervous system tumor microenvironment. Clin Cancer Res, 2005. 11(15): p. 5515-25.

109. Walker, D. G., et al., Results of a phase I dendritic cell vaccine trial for malignant astrocytoma: potential interaction with adjuvant chemotherapy. J Clin Neurosci, 2008. 15(2): p. 114-21.

110. Leplina, O. Y., et al., Use of interferon-alpha-induced dendritic cells in the therapy of patients with malignant brain gliomas. Bull Exp Biol Med, 2007. 143(4): p. 528-34.

111. De Vleeschouwer, S., et al., Postoperative adjuvant dendritic cell-based immunotherapy in patients with relapsed glioblastoma multiforme. Clin Cancer Res, 2008. 14(10): p. 3098-104.

112. Ardon, H., et al., Adjuvant dendritic cell-based tumour vaccination for children with malignant brain tumours. Pediatr Blood Cancer, 2010. 54(4): p. 519-25.

113. Prins, R. M., et al., Gene expression profile correlates with T-cell infiltration and relative survival in glioblastoma patients vaccinated with dendritic cell immunotherapy. Clin Cancer Res, 2011. 17(6): p. 1603-15.

114. Okada, H., et al., Induction of CD8+ T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. J Clin Oncol, 2011. 29(3): p. 330-6.

115. Fadul, C. E., et al., Immune response in patients with newly diagnosed glioblastoma multiforme treated with intranodal autologous tumor lysate-dendritic cell vaccination after radiation chemotherapy. J Immunother, 2011. 34(4): p. 382-9.

116. Chang, C. N., et al., A phase I/II clinical trial investigating the adverse and therapeutic effects of a postoperative autologous dendritic cell tumor vaccine in patients with malignant glioma. J Clin Neurosci, 2011. 18(8): p. 1048-54.

117. Cho, D. Y., et al., Adjuvant immunotherapy with whole-cell lysate dendritic cells vaccine for glioblastoma multiforme: a phase II clinical trial. World Neurosurg, 2012. 77(5-6): p. 736-44.

118. Iwami, K., et al., Peptide-pulsed dendritic cell vaccination targeting interleukin-13 receptor alpha2 chain in recurrent malignant glioma patients with HLA-A*24/A*02 allele. Cytotherapy, 2012. 14(6): p. 733-42.

119. Fong, B., et al., Monitoring of regulatory T cell frequencies and expression of CTLA-4 on T cells, before and after DC vaccination, can predict survival in GBM patients. PLoS One, 2012. 7(4): p. e32614.

120. De Vleeschouwer, S., et al., Stratification according to HGG-IMMUNO RPA model predicts outcome in a large group of patients with relapsed malignant glioma treated by adjuvant postoperative dendritic cell vaccination. Cancer Immunol Immunother, 2012. 61(11): p. 2105-12.

121. Phuphanich, S., et al., Phase I trial of a multi-epitope-pulsed dendritic cell vaccine for patients with newly diagnosed glioblastoma. Cancer Immunol Immunother, 2013. 62(1): p. 125-35.

122. Akiyama, Y., et al., alpha-type-1 polarized dendritic cell-based vaccination in recurrent high-grade glioma: a phase I clinical trial. BMC Cancer, 2012. 12: p. 623.

123. Prins, R. M., et al., Comparison of glioma-associated antigen peptide-loaded versus autologous tumor lysate-loaded dendritic cell vaccination in malignant glioma patients. J Immunother, 2013. 36(2): p. 152-7.

124. Shah, A. H., et al., Dendritic cell vaccine for recurrent high-grade gliomas in pediatric and adult subjects: clinical trial protocol. Neurosurgery, 2013. 73(5): p. 863-7.

125. Reichardt, V. L., et al., Idiotype vaccination using dendritic cells after autologous peripheral blood stem cell transplantation for multiple myeloma—a feasibility study. Blood, 1999. 93(7): p. 2411-9.

126. Lim, S. H. and R. Bailey-Wood, Idiotypic protein pulsed dendritic cell vaccination in multiple myeloma. Int J Cancer, 1999. 83(2): p. 215-22.

127. Motta, M. R., et al., Generation of dendritic cells from CD14+monocytes positively selected by immunomagnetic adsorption for multiple myeloma patients enrolled in a clinical trial of anti-idiotype vaccination. Br J Haematol, 2003. 121(2): p. 240-50.

128. Reichardt, V. L., et al., Idiotype vaccination of multiple myeloma patients using monocyte-derived dendritic cells. Haematologica, 2003. 88(10): p. 1139-49.

129. Guardino, A. E., et al., Production of myeloid dendritic cells (DC) pulsed with tumor-specific idiotype protein for vaccination of patients with multiple myeloma. Cytotherapy, 2006. 8(3): p. 277-89.

130. Lacy, M. Q., et al., Idiotype-pulsed antigen presenting cells following autologous transplantation for multiple myeloma may be associated with prolonged survival. Am J Hematol, 2009. 84(12): p. 799-802.

131. Yi, Q., et al., Optimizing dendritic cell-based immunotherapy in multiple myeloma: intranodal injections of idiotype-pulsed CD40 ligand-matured vaccines led to induction of type-1 and cytotoxic T-cell immune responses in patients. Br J Haematol, 2010. 150(5): p. 554-64.

132. Rollig, C., et al., Induction of cellular immune responses in patients with stage-I multiple myeloma after vaccination with autologous idiotype-pulsed dendritic cells. J Immunother, 2011. 34(1): p. 100-6.

133. Zahradova, L., et al., Efficacy and safety of Id-protein-loaded dendritic cell vaccine in patients with multiple myeloma—phase II study results. Neoplasma, 2012. 59(4): p. 440-9.

134. Timmerman, J. M., et al., Idiotype pulsed dendritic cell vaccination for B-cell lymphoma: clinical and immune responses in 35 patients. Blood, 2002. 99(5): p. 1517-26.

135. Maier, T., et al., Vaccination of patients with cutaneous T-cell lymphoma using intranodal injection of autologous tumor-lysate-pulsed dendritic cells. Blood, 2003. 102(7): p. 2338-44.

136. Di Nicola, M., et al., Vaccination with autologous tumor-loaded dendritic cells induces clinical and immunologic responses in indolent B-cell lymphoma patients with relapsed and measurable disease: a pilot study. Blood, 2009. 113(1): p. 18-27.

137. Hus, I., et al., Allogeneic dendritic cells pulsed with tumor lysates or apoptotic bodies as immunotherapy for patients with early-stage B-cell chronic lymphocytic leukemia. Leukemia, 2005. 19(9): p. 1621-7.

138. Li, L., et al., Immunotherapy for patients with acute myeloid leukemia using autologous dendritic cells generated from leukemic blasts. Int J Oncol, 2006. 28(4): p. 855-61.

139. Roddie, H., et al., Phase I/II study of vaccination with dendritic-like leukaemia cells for the immunotherapy of acute myeloid leukaemia. Br J Haematol, 2006. 133(2): p. 152-7.

140. Litzow, M. R., et al., Testing the safety of clinical-grade mature autologous myeloid DC in a phase I clinical immunotherapy trial of CML. Cytotherapy, 2006. 8(3): p. 290-8.

141. Westermann, J., et al., Vaccination with autologous non-irradiated dendritic cells in patients with bcr/abl+chronic myeloid leukaemia. Br J Haematol, 2007. 137(4): p. 297-306.

142. Hus, I., et al., Vaccination of B-CLL patients with autologous dendritic cells can change the frequency of leukemia antigen-specific CD8+ T cells as well as CD4+CD25+FoxP3+regulatory T cells toward an antileukemia response. Leukemia, 2008. 22(5): p. 1007-17.

143. Palma, M., et al., Development of a dendritic cell-based vaccine for chronic lymphocytic leukemia. Cancer Immunol Immunother, 2008. 57(11): p. 1705-10.

144.Van Tendeloo, V. F., et al., Induction of complete and molecular remissions in acute myeloid leukemia by Wilms' tumor 1 antigen-targeted dendritic cell vaccination. Proc Natl Acad Sci USA, 2010. 107(31): p. 13824-9.

145. Iwashita, Y., et al., A phase I study of autologous dendritic cell-based immunotherapy for patients with unresectable primary liver cancer. Cancer Immunol Immunother, 2003. 52(3): p. 155-61.

146. Lee, W. C., et al., Vaccination of advanced hepatocellular carcinoma patients with tumor lysate-pulsed dendritic cells: a clinical trial. J Immunother, 2005. 28(5): p. 496-504.

147. Butterfield, L. H., et al., A phase I/II trial testing immunization of hepatocellular carcinoma patients with dendritic cells pulsed with four alpha-fetoprotein peptides. Clin Cancer Res, 2006. 12(9): p. 2817-25.

148. Palmer, D. H., et al., A phase II study of adoptive immunotherapy using dendritic cells pulsed with tumor lysate in patients with hepatocellular carcinoma. Hepatology, 2009. 49(1): p. 124-32.

149. El Ansary, M., et al., Immunotherapy by autologous dendritic cell vaccine in patients with advanced HCC. J Cancer Res Clin Oncol, 2013. 139(1): p. 39-48.

150. Tada, F., et al., Phase I/II study of immunotherapy using tumor antigen-pulsed dendritic cells in patients with hepatocellular carcinoma. Int J Oncol, 2012. 41(5): p. 1601-9.

151. Ueda, Y., et al., Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. Int J Oncol, 2004. 24(4): p. 909-17.

152. Hirschowitz, E. A., et al., Autologous dendritic cell vaccines for non-small-cell lung cancer. J Clin Oncol, 2004. 22(14): p. 2808-15.

153. Chang, G. C., et al., A pilot clinical trial of vaccination with dendritic cells pulsed with autologous tumor cells derived from malignant pleural effusion in patients with late-stage lung carcinoma. Cancer, 2005. 103(4): p. 763-71.

154. Yannelli, J. R., et al., The large scale generation of dendritic cells for the immunization of patients with non-small cell lung cancer (NSCLC). Lung Cancer, 2005. 47(3): p. 337-50.

155. Ishikawa, A., et al., A phase I study of alpha-galactosylceramide (KRN7000)-pulsed dendritic cells in patients with advanced and recurrent non-small cell lung cancer. Clin Cancer Res, 2005. 11(5): p. 1910-7.

156. Antonia, S. J., et al., Combination of p53 cancer vaccine with chemotherapy in patients with extensive stage small cell lung cancer. Clin Cancer Res, 2006. 12(3 Pt 1): p. 878-87.

157. Perrot, I., et al., Dendritic cells infiltrating human non-small cell lung cancer are blocked at immature stage. J Immunol, 2007. 178(5): p. 2763-9.

158. Hirschowitz, E. A., et al., Immunization of NSCLC patients with antigen-pulsed immature autologous dendritic cells. Lung Cancer, 2007. 57(3): p. 365-72.

159. Baratelli, F., et al., Pre-clinical characterization of GMP grade CCL21-gene modified dendritic cells for application in a phase I trial in non-small cell lung cancer. J Transl Med, 2008. 6: p. 38.

160. Hegmans, J. P., et al., Consolidative dendritic cell-based immunotherapy elicits cytotoxicity against malignant mesothelioma. Am J Respir Crit Care Med, 2010. 181(12): p. 1383-90.

161. Um, S. J., et al., Phase I study of autologous dendritic cell tumor vaccine in patients with non-small cell lung cancer. Lung Cancer, 2010. 70(2): p. 188-94.

162. Chiappori, A. A., et al., INGN-225: a dendritic cell-based p53 vaccine (Ad.p53-DC) in small cell lung cancer: observed association between immune response and enhanced chemotherapy effect. Expert Opin Biol Ther, 2010. 10(6): p. 983-91.

163. Perroud, M. W., Jr., et al., Mature autologous dendritic cell vaccines in advanced non-small cell lung cancer: a phase I pilot study. J Exp Clin Cancer Res, 2011. 30: p. 65.

164. Skachkova, O. V., et al., Immunological markers of anti-tumor dendritic cells vaccine efficiency in patients with non-small cell lung cancer. Exp Oncol, 2013. 35(2): p. 109-13.

165. Hernando, J. J., et al., Vaccination with autologous tumour antigen pulsed dendritic cells in advanced gynaecological malignancies: clinical and immunological evaluation of a phase I trial. Cancer Immunol Immunother, 2002. 51(1): p. 45-52.

166. Rahma, O. E., et al., A gynecologic oncology group phase II trial of two p53 peptide vaccine approaches: subcutaneous injection and intravenous pulsed dendritic cells in high recurrence risk ovarian cancer patients. Cancer Immunol Immunother, 2012. 61(3): p. 373-84.

167. Chu, C. S., et al., Phase I/II randomized trial of dendritic cell vaccination with or without cyclophosphamide for consolidation therapy of advanced ovarian cancer in first or second remission. Cancer Immunol Immunother, 2012. 61(5): p. 629-41.

168. Kandalaft, L. E., et al., A Phase I vaccine trial using dendritic cells pulsed with autologous oxidized lysate for recurrent ovarian cancer. J Transl Med, 2013. 11: p. 149.

169. Lepisto, A. J., et al., A phase I/II study of a MUC1 peptide pulsed autologous dendritic cell vaccine as adjuvant therapy in patients with resected pancreatic and biliary tumors. Cancer Ther, 2008. 6(B): p. 955-964.

170. Rong, Y., et al., A phase I pilot trial of MUC1-peptide-pulsed dendritic cells in the treatment of advanced pancreatic cancer. Clin Exp Med, 2012. 12(3): p. 173-80.

171. Endo, H., et al., Phase I trial of preoperative intratumoral injection of immature dendritic cells and OK-432 for resectable pancreatic cancer patients. J Hepatobiliary Pancreat Sci, 2012. 19(4): p. 465-75.

172. Tsujitani, S., et al., Infiltration of dendritic cells in relation to tumor invasion and lymph node metastasis in human gastric cancer. Cancer, 1990. 66(9): p. 2012-6.

173. Tsujitani, S., et al., Postoperative adjuvant immunochemotherapy and infiltration of dendritic cells for patients with advanced gastric cancer. Anticancer Res, 1992. 12(3): p. 645-8.

174. Kakeji, Y., et al., Prognostic significance of tumor-host interaction in clinical gastric cancer: relationship between DNA ploidy and dendritic cell infiltration. J Surg Oncol, 1993. 52(4): p. 207-12.

175. Ishigami, S., et al., Prognostic value of HLA-DR expression and dendritic cell infiltration in gastric cancer. Oncology, 1998. 55(1): p. 65-9.

176. Giannini, A., et al., Prognostic significance of accessory cells and lymphocytes in nasopharyngeal carcinoma. Pathol Res Pract, 1991. 187(4): p. 496-502.

177. Gallo, O., et al., Correlations between histopathological and biological findings in nasopharyngeal carcinoma and its prognostic significance. Laryngoscope, 1991. 101(5): p. 487-93.

178. Furihata, M., et al., HLA-DR antigen- and S-100 protein-positive dendritic cells in esophageal squamous cell carcinoma—their distribution in relation to prognosis. Virchows Arch B Cell Pathol Incl Mol Pathol, 1992. 61(6): p. 409-14.

179. Reichert, T. E., et al., The number of intratumoral dendritic cells and zeta-chain expression in T cells as prognostic and survival biomarkers in patients with oral carcinoma. Cancer, 2001. 91(11): p. 2136-47.

180. Timar, J., et al., Neoadjuvant immunotherapy of oral squamous cell carcinoma modulates intratumoral CD4/CD8 ratio and tumor microenvironment. a multicenter phase II clinical trial. J Clin Oncol, 2005. 23(15): p. 3421-32.

181. Nakano, T., et al., Antitumor activity of Langerhans cells in radiation therapy for cervical cancer and its modulation with SPG administration. In Vivo, 1993. 7(3): p. 257-63.

182. Lespagnard, L., et al., Tumor-infiltrating dendritic cells in adenocarcinomas of the breast: a study of 143 neoplasms with a correlation to usual prognostic factors and to clinical outcome. Int J Cancer, 1999. 84(3): p. 309-14.

183. Iwamoto, M., et al., Prognostic value of tumor-infiltrating dendritic cells expressing CD83 in human breast carcinomas. Int J Cancer, 2003. 104(1): p. 92-7.

184. Coventry, B. J. and J. Morton, CD1a-positive infiltrating-dendritic cell density and 5-year survival from human breast cancer. Br J Cancer, 2003. 89(3): p. 533-8.

185. Zhao, R., et al., [Study on the relationship between the dendritic cell infiltration in cancer tissues and prognosis in patients with lung cancer]. Zhongguo Fei Ai Za Zhi, 2002. 5(2): p. 112-4.

186. Diederichsen, A. C., et al., Prognostic value of the CD4+/CD8+ ratio of tumour infiltrating lymphocytes in colorectal cancer and HLA-DR expression on tumour cells. Cancer Immunol Immunother, 2003. 52(7): p. 423-8.

187. Dadabayev, A. R., et al., Dendritic cells in colorectal cancer correlate with other tumor-infiltrating immune cells. Cancer Immunol Immunother, 2004. 53(11): p. 978-86.

188. Sandel, M. H., et al., Prognostic value of tumor-infiltrating dendritic cells in colorectal cancer: role of maturation status and intratumoral localization. Clin Cancer Res, 2005. 11(7): p. 2576-82.

189. Yin, X. Y., et al., Prognostic significances of tumor-infiltrating S-100 positive dendritic cells and lymphocytes in patients with hepatocellular carcinoma. Hepatogastroenterology, 2003. 50(53): p. 1281-4.

190. Cal, X. Y., et al., Dendritic cell infiltration and prognosis of human hepatocellular carcinoma. J Cancer Res Clin Oncol, 2006. 132(5): p. 293-301.

191. Nakakubo, Y., et al., Clinical significance of immune cell infiltration within gallbladder cancer. Br J Cancer, 2003. 89(9): p. 1736-42.

192. Furihata, M., et al., Prognostic significance of CD83 positive, mature dendritic cells in the gallbladder carcinoma. Oncol Rep, 2005. 14(2): p. 353-6.

193. Fukunaga, A., et al., CD8+ tumor-infiltrating lymphocytes together with CD4+ tumor-infiltrating lymphocytes and dendritic cells improve the prognosis of patients with pancreatic adenocarcinoma. Pancreas, 2004. 28(1): p. e26-31.

194. Ugel, S., et al., Targeting tumor vasculature: expanding the potential of DNA cancer vaccines. Cancer Immunol Immunother, 2015. 64(10): p. 1339-48.

195. Li, L., et al., Developing a clinical development paradigm for translation of a mammaglobin-A DNA vaccine. Immunotherapy, 2015: p. 1-3.

196. Tiriveedhi, V., et al., Safety and preliminary evidence of biologic efficacy of a mammaglobin-a DNA vaccine in patients with stable metastatic breast cancer. Clin Cancer Res, 2014. 20(23): p. 5964-75.

197. Heller, R. and L. C. Heller, Gene electrotransfer clinical trials. Adv Genet, 2015. 89: p. 235-62.

What is claimed is:

1. A composition comprising fibroblasts, wherein the fibroblasts are manipulated to express or produce one or more oncogenes and/or tumor antigens and/or genes that induce the expression of one or more oncogenes and/or tumor antigens.

2. The composition of claim 1, wherein said oncogenes are selected from the group consisting of hTERT, c-myc, k-RAS, CTCFL, AF4/HRX, ABL, ALK, AKT-2, ALK/NPM, AML1, AML1/MTG8, AXL, BCL-2, BCL-3, BCL-6, BCL-XL, BCR/ABL, DBL, DEK/CAN, E2A/PBX1, EGFR, ENL/HRX, ERG/TLS, ERBB, ERBB-2, ETS-1, EWS/FLI-1, FMS, FOS, FPS, GLI, GSP, HOX11, HER2/neu, HST, INT-2, JUN, KIT, KS3, K-SAM, LBC, LCK, LM02/LM01, LYL-1, LYT-10, TSK, TRK, and a combination thereof.

3. The composition of claim 1, wherein said tumor specific antigen(s) is selected from the group of consisting of Fos-related antigen 1, LCK, FAP, VEGFR2, NA17, PDGFR-beta, PAP, MAD-CT-2, Tie-2, PSA, protamine 2, legumain, endosialin, prostate stem cell antigen, carbonic anhydrase IX, STn, Page4, proteinase 3, GM3 ganglioside, tyrosinase, MART1, gp100, SART3, RGS5, SSX2, Globo11, Tn, CEA, hCG, PRAME, XAGE-1, AKAP-4, TRP-2, B7H3, sperm fibrous sheath protein, CYP1B1, HMWMAA, sLe(a), MAGE A1, GD2, PSMA, mesothelin, fucosyl GM1, GD3, sperm protein 17, NY-ESO-1, PAX5, AFP, polysialic acid, EpCAM, MAGE-A3, mutant p53, ras, mutant ras, NY-BR1, PAX3, HER2/neu, OY-TES1, HPV E6 E7, PLAC1, hTERT, BORIS, ML-IAP, idiotype of b cell lymphoma or multiple myeloma, EphA2, EGFRvIII, cyclin B 1, RhoC, androgen receptor, surviving, MYCN, wildtype p53, LMP2, ETV6-AML, MUC1, BCR-ABL, ALK, WT1, ERG (TMPRSS2 ETS fusion gene), sarcoma translocation breakpoint, STEAP, OFA/iLRP, Chondroitin sulfate proteoglycan 4 (CSPG4), alphaGal, and a combination thereof.

4. The composition of claim 1, wherein the gene that induces the expression of one or more oncogenes and/or tumor suppressors is selected from the group consisting of TRAIL, TNF-alpha, interferon gamma, interferon alpha, interferon beta, IL-12, IL-18, IL-21, IL-28, alpha-1,3-galactosyltransferase, and a combination thereof.

5. The composition of any one of claims 1-4, wherein the manipulation is further defined as exogenous expression of the one or more tumor antigens and/or oncogenes and/or genes that induce the expression of one or more oncogenes and/or tumor antigens.

6. The composition of any one of claims 1-5, wherein the manipulation comprises exposure to one or more conditions that result in dedifferentiation of the fibroblasts.

7. The composition of any one of claims 1-6, wherein said fibroblasts are manipulated by culturing under conditions of hypoxia.

8. The composition of claim 7, wherein said hypoxia comprises culture in an oxygen tension that ranges from 0.1%-10% oxygen.

9. The composition of claim 7, wherein the cells are cultured in hypoxia for approximately 1, 5, 10, 15, 20, 25, 30, 35, 40, 45, 50, 55, 60 minutes, or any time from 1-60 minutes.

10. The composition of claim 7 wherein the cells are culture in hypoxia for approximately 1, 2, 3, 4, 5, 6, 7, 8, 9, 10, 11, 12, 13, 14, 15, 16, 17, 18, 19, 20, 21, 22, 23, or 24 hours.

11. The composition of claim 7, wherein the cells are cultured in hypoxia for approximately 1, 2, 3, 4, 5, or more days.

12. The composition of claim 7, wherein said fibroblast is cultured at approximately 3% oxygen for approximately 24 hours.

13. The composition of any one of claims 1-12, wherein the fibroblasts are manipulated by exposure one or more dedifferentiation signals.

14. The composition of claim 13, wherein the fibroblasts are exposed to said dedifferentiation signal(s) for a sufficient time period and at a sufficient concentration to induce expression of oncogenes in said fibroblasts.

15. The composition of claim 13, wherein said dedifferentiation signal(s) are administered for a sufficient time period and at a sufficient concentration to induce expression of tumor antigens in said fibroblasts.

16. The composition of any one of claims 13-15, wherein said dedifferentiation is accomplished through incubation in acidic conditions.

17. The composition of any one of claims 13-16, wherein said dedifferentiation is accomplished through incubation in cytoplasm of cells that are in a dedifferentiated state.

18. The composition of any one of claims 13-17, wherein said dedifferentiation is accomplished through incubation in cytoplasm of pluripotent cells.

19. The composition of claim 18, wherein said pluripotent stem cells are selected from the group consisting of inducible pluripotent stem cells, placental stem cells, parthenogenic derived stem cells, and a combination thereof.

20. The composition of any one of claims 13-19, wherein said dedifferentiation is performed by culture in the presence of a histone deacetylase inhibitor.

21. The composition of any one of claims 13-20, wherein said dedifferentiation is performed by culture in the presence of a DNA methyltransferase inhibitor.

22. The composition of any one of claims 13-21, wherein said dedifferentiation is performed by culture in the presence of a GSK-3 inhibitor.

23. The composition of any one of claims 1-22, wherein the fibroblasts are manipulated to express one or more oncogenes and/or tumor antigens and/or genes that induces the expression of one or more oncogenes and/or tumor antigens comprising the step of
transfecting, transducing, electroporating, transforming a vector encoding one or more oncogenes and/or tumor antigens into the fibroblasts; or
pulsing a peptide or fragment of one or more oncogenes and/or tumor antigens into the fibroblasts.

24. The composition of claim 23, wherein the vector is inducible by a hypoxia-inducible promoter, an acid inducible promoter, or both.

25. The composition of claim 23, wherein the vector is inducible by a drug inducible promoter.

26. The composition of any one of claims 1-25, wherein the fibroblasts are manipulated to express one or more oncogenes and/or tumor antigens and/or genes that induce the expression of one or more oncogenes and/or tumor antigens, comprising the step of transfecting, transducing, electroporating, transforming a vector encoding one or more immune stimulatory genes into the fibroblasts, wherein at least one of the immune stimulatory genes, when expressed in the fibroblast, induces the expression and/or translocation of at least one oncogene and/or tumor antigen onto the surface of the fibroblast.

27. The composition of claim 26, wherein the vector is inducible by a hypoxia-inducible promoter, acid inducible promoter, or both.

28. The composition of claim 26, wherein the vector is inducible by a drug inducible promoter, an acid inducible promoter, or both.

29. The composition of claim 26, wherein the immune stimulatory gene is selected from the group consisting of TRAIL, TNF-alpha, interferon gamma, interferon alpha, interferon beta, IL-12, IL-18, IL-21, IL-28, alpha-1,3-galactosyltransferase, and a combination thereof.

30. The composition of any one of claims 1-29, wherein the fibroblasts possess the ability to selectively migrate towards cancers.

31. The composition of any one of claims 1-30, wherein the fibroblast expresses CXCR4.

32. The method of any one of claims 1-31, wherein said fibroblast population is from a tissue selected from the group consisting of omentum, bone marrow, placenta, peripheral blood, cord blood, Wharton's jelly, cerebral spinal fluid, cancer associated, foreskin, skin, and a combination thereof.

33. A method of inducing an immune response against cancer in an individual, comprising the step of administering a composition of any one of claims 1-32 to the individual.

34. The method of claim 33, wherein the composition is administered therapeutically.

35. The method of claim 33, wherein the composition is administered prophylactically.

36. The method of any one of claims 33-35, wherein the composition in administered systemically.

37. The method of any one of claims 33-35, wherein the composition in administered peritumorally to the cancer affecting the individual.

38. The method of any one of claims 33-37, wherein the composition in administered locally to the cancer affecting the individual.

39. The method of any one of claims 33-38, wherein the composition is administered in combination with at least one composition that induces T-cell activation.

40. The method of claim 39, wherein the composition that induces T-cell activitation comprises one or more co-stimulatory agonists.

41. The method of claim 40, wherein the co-stimulatory agonist comprises an agonistic antibody against a co-stimulatory molecule selected from the group consisting of: CD28, OX40, GITR, CD137, CD27, HVEM, and a combination thereof.

42. The method of claim 39, wherein the composition that induces T-cell activitation is one or more co-inhibitory antagonists.

43. The method of claim 42, wherein the co-inhibitory antagonist comprises an antagonistic antibody against a negative co-stimulatory molecule selected from the group consisting of CTLA-4, PD-1, PDL-1, and a combination thereof.

44. The method of claim 42, wherein the co-inhibitory antagonist comprises an antagonist that inhibits the function of CTLA-4, PD-1, PDL-1, AMP-244, MEDI-4736, MPDL328 OA, MIH1, or a combination thereof.

45. The method of any one of claims 33-44 , wherein said administering results in regression of tumor.

46. The method of any one of claims 33-45, wherein said administering results in complement activation in the tumor or in proximity of the tumor.

47. The method of any one of claims 33-46, wherein said administering results in T cell activation in the tumor or in proximity of the tumor.

48. The method of any one of claims 33-47, wherein said administering results in NK cell activation in the tumor or in proximity of the tumor.

49. The method of any one of claims 33-48, wherein said administering results in M1 cell activation in the tumor or in proximity of the tumor.

50. The method of any one of claims 33-49, wherein said administering results in suppression of M2 cell activation in the tumor or in proximity of the tumor.

51. The method of any one of claims 33-50, wherein said administering results in suppression of angiogenesis inside the tumor.

52. The method of any one of claims 33-51, wherein said administering results in generation of antibodies specific to the tumor.

53. The method of any one of claims 33-52, wherein said administering results in activation of antibody dependent cellular cytotoxicity in the tumor or in proximity to the tumor.


SEQUENCE LISTING (2 sequences)

SEQ ID NO: 1 - DNA, 25 bp, Artificial Sequence
  Description of Artificial Sequence Synthetic primer
agtagtaggtttctgttctattggg

SEQ ID NO: 2 - DNA, 26 bp, Artificial Sequence
  Description of Artificial Sequence Synthetic primer
tacttattccactgctgttattatcc